(19) United States
(12) Patent Application Publication
(10) Pub. No.: US 2009/0311249 A1
(43) Pub. Date: Dec. 17, 2009

(54) Capecitabine Combination Therapy

(76) Inventors: Luca Gianni, Milan (IT); Camille L. Bedrosian, Belmont, MA (US)

Correspondence Address: ARIAD PHARMACEUTICALS, INC.;ARIAD GENE THERAPEUTICS, INC., 26 LANDSDOWNE ST., CAMBRIDGE, MA, 02139, US

(21) Appl. No.: 12/227,656

(22) PCT Filed: Jun. 4, 2007
(86) PCT No.: PCT/US2007/013243

§ 371 Date: Apr. 13, 2009

Related U.S. Application Data
(60) Provisional application No. 60/810,371, filed on Jun. 2, 2006.

(51) Int. Cl. A61K 39/395; A61P 35/00; A61K 31/506

(52) U.S. Cl. 424/133.1; 514/274

(57) ABSTRACT

The invention provides the use of a combination of an mTOR inhibitor and capecitabine in the treatment of cancer.

### BACKGROUND OF THE INVENTION

Cancer is reportedly the second leading cause of death in the United States and, if current trends continue, may become the leading cause of death by 2010.

Currently, a variety of drugs with different mechanisms of action are available for the treatment of cancer. Some act by inhibiting DNA synthesis, either directly, or indirectly by inhibiting the biosynthesis of the deoxyribonucleotide precursors, to prevent DNA replication and concomitant cell division. These drugs, which include alkylating agents and antimetabolites, although not necessarily cell cycle specific, generally kill cells during their S phase because of their effect on DNA replication. Other chemotherapeutic agents, such as the taxanes and vinca alkaloids, interfere with microtubule assembly, resulting in mitotic arrest.

New therapeutics with various mechanisms of action are continuously developed for possible inclusion in the arsenal of drugs for the treatment of cancer. For example, several compounds that inhibit mTOR, a serine-threonine kinase involved in the PI3K/Akt signaling pathway, have been demonstrated to exhibit anti-cancer properties. The PI3K/Akt pathway, which participates in the regulation of multiple biological phenomema such as control of transcription and translation of certain proteins, is thought to be over-activated in numerous cancers. mTOR inhibitors that have been shown to be promising agents for treating certain cancers include rapamycin (sirolimus) and rapamycin derivatives, such as ARIAD's AP23573, Wyeth's CCI-779 (temsirolimus) and Novartis' RAD001 (SDZ RAD, everolimus, Certican™).

Despite the availability of a variety of chemotherapeutic agents, significant challenges persist. Most chemotherapeutic agents approved to date exhibit profound and often dangerous side effects including immunosuppression, bone marrow depression, severe nausea, etc., which can be dose limiting. In order to increase the efficacy of cancer treatment, some protocols involve administration of a combination of two or more anti-cancer drugs. Drugs with different mechanisms of action have been considered for use in combination therapy, based on the rationale that targeting multiple different cellular pathways may result in enhanced efficacy, and in some cases, supported by promising in vitro data. However, combining drugs can also compound side effect issues by combining the drugs' respective toxicities. While some combinations of chemotherapeutic agents have led to positive clinical results, others have unfortunately proved simply too toxic for human patients, notwithstanding theoretical advantages or intriguing results on isolated cells grown in the lab.

As a case in point, some have suggested combining mTOR inhibitors and antimetabolite drugs for the treatment of cancer (see, for example, U.S. Pat. No. 5,206,018; WO 02/066019 and US Pat. Appln. No. 2004/0145741; U.S. Pat. No. 7,091,213; WO 02/080975 and US Pat. Appln. Nos. 2002-0183239 and 2006-0035904; and 2002-0183240 and 2005-0187184). However, clinical trials with combinations of mTOR inhibitors and antimetabolites have revealed serious toxicities. In particular, Phase I clinical trials using temsirolimus in combination with 5-fluorouracil (5FU) and leuvocorin for the treatment of patients with advanced solid tumors have been terminated for unacceptable toxicity (C. J. Punt et al., Ann. Oncol., 2003, 14: 931-937). Similarly, Phase I clinical trials using everolimus and gemcitabine in patients with advanced cancers (S. Pacey et al., J. Clin. Oncol., 2004 ASCO Annual Meeting Proceedings (Post-Meeting Edition), Vol. 22, No. 14S (July 15 Supplement): 3120) have been stopped because a majority of patients could not tolerate the combination therapy.

### SUMMARY OF THE INVENTION

This invention is based on the surprising clinical finding that—despite the discouraging prior clinical studies of combinations of mTOR inhibitors with the antimetabolites noted above—co-administration of an mTOR inhibitor with the antimetabolite, capecitabine, can be used to treat cancer patients without causing unacceptable toxicity.

This invention thus provides, in one aspect, a method for treating cancer in a patient by co-administering to the patient an mTOR inhibitor and capecitabine. Treatment effective amounts of mTOR inhibitors suitable for use in this method are discussed below. The capecitabine is typically administered in a repeating cycle of total daily doses of 1000-2500 mg of capecitabine/m² p.o. (orally) each day for 7-14 days every 21-28 days. The daily dose of capecitabine is typically divided into two portions, e.g., of 500-1250 mg/m², given at different times of day, e.g., about 12 hours apart, followed by a period of 7-14 days without capecitabine treatment. The mTOR inhibitor can be given before, after or simultaneously with the capecitabine, and on the same or different dosing schedules and by the same or different routes of administration.

As discussed in greater detail below, this invention also provides products and kits containing an mTOR inhibitor and capecitabine in formulations permitting their simultaneous, separate or sequential administration to patients. The materials and methods of this invention may be used in the full range of relevant therapeutic situations, including, e.g., neo-adjuvant, adjuvant, maintenance and salvage contexts.

Non-limiting examples of mTOR inhibitors for use in practicing this invention include rapamycin and rapamycin analogs, and may be administered by any pharmaceutically acceptable route, a variety of which are known for that class of drugs. Oral and parenteral (e.g., i.v.) administration are currently of particular interest. The mTOR inhibitors of greatest current interest are rapamycin analogs in which the hydroxyl group at position 43 is replaced, especially those analogs currently in clinical development for treating cancer, such as AP23573, everolimus and temsirolimus. At present, parenteral administration is of particular interest in the case of temsirolimus, oral administration for everolimus, and either route for AP23573. These and other mTOR inhibitors are discussed in greater detail below.

Dose levels for the mTOR inhibitor in this combination therapy are generally in the range of 10-800 mg overall per week of treatment, e.g., in some cases 35-250 mg/week. Such overall weekly dosage levels may be achieved using a variety of routes of administration and dosing schedules.

The dosing schedule may be intermittent. “Intermittent” dosing refers to schedules providing intervening periods between doses, e.g. every second day dosing, every third day dosing, or more generally, schedules containing “holidays” of one or more days or weeks between periods of dosing. Non-limiting examples of such intermittent dosing including dosing on fewer than seven days per week as well as dosing cycles of one week of QDx4, QDx5, QDx6 or daily dosing followed by a period without drug, e.g., one, two or three weeks, then resuming with another week of drug treatment followed by a week (or weeks) without drug treatment, and so on. To illustrate further, administration of 60 mg QDx6 every other week provides a weekly dose of 360 mg of drug on an intermittent basis (i.e., every other week).

For example, in the case of oral administration, 2-160 mg of the drug can be given one or more days per week, e.g. every day (QDx7), six days per week (QDx6), five days per week (QDx5), etc. Thus, Everolimus may be given QDx7 at doses of 3-20 mg/day, e.g., 5 mg or 10 mg. AP23573 may be given QDx7 p.o. at doses of 10-25 mg/day, e.g., 10, 12.5 or 15 mg/day; or sirolimus at 2 or 4 mg p.o. QDx7, in some cases with a 6, 8 or 10 mg loading dose. The dosing schedule may be intermittent, as illustrated by QDx4, QDx5 and QDx6 schedules. Examples include oral administration of the mTOR inhibitor at 30-100 mg QDx5 or QDx6. For instance, in the practice of this invention, AP23573, everolimus, temsirolimus or sirolimus is administered orally at levels of 10-50 mg QDx5. Of current interest are QDx5 dose levels of 30-50 mg p.o., and in the case of AP23573, QDx5 dose levels of 30 or 40 mg p.o. are of particular interest.

The desired overall level of exposure to the drug can alternatively be achieved by various schedules of parenteral delivery. In such cases, 10-250 mg of the mTOR inhibitor is administered, for example, by i.v. infusion over 15-60 minutes, often 30-60 minutes, one or more times per 1- to 4-week period.

In one such approach, the mTOR inhibitor is administered in a 30-60 minute i.v. infusion once each week for three or four weeks every 4-week cycle. Such i.v. delivery is of particular interest in the case of AP23573, sirolimus and temsirolimus, which can be provided, for example, in weekly doses of 10-250 mg, e.g., 25, 50, 75, 100, 150, 200 or 250 mg/week, for three or four weeks of each 4-week cycle. Dose levels of 50 and 75 mg are of particular current interest.

In another approach, the mTOR inhibitor is administered by iv infusion of 5-25 mg of the drug QDx5 every two weeks (e.g., with iv infusions Monday through Friday, every 2d week). Doses of 10, 12.5, 15, 17.5 and 20 mg are of particular current interest.

Of interest are dose levels and dosing schedules already approved or under study for rapamycin or the analog, in a monotherapy or other drug combination regimen.

In one embodiment of the invention, either or both of the mTOR inhibitor and the capecitabine are administered intravenously. In other cases, either or both are administered orally. For example, AP23573 may be administered intravenously, e.g., using a 30-60 minute infusion, and capecitabine may be administered orally. Alternatively, both agents may be administered orally. Illustrative examples of co-administration regimens are set forth in the table below:

Illustrative Combination Regimens
Capecitabine: 1000-2500 mg/m²/day orally (split into two doses)
for 14 days followed by 7-14 days without capecitabine
Plus one of the following mTOR inhibitors:

| | |
|---|---|
| AP23573: | 10 or 15 mg daily, orally (QD×7, po) |
| AP23573: | 30-40 mg daily, orally, 5 days/week (QD×5) |
| AP23573: | 12.5 mg via i.v. infusion QD×5 every 2 d week |
| Everolimus: | 5 or 10 mg (QD×7, po) |
| Temsirolimus: | 15 or 19 mg/m²/day i.v. infusion for 5 days, every |
| | 2 d week |
| Temsirolimus: | 25 mg i.v. infusion once per week |

Several examples below illustrate staggered drug administration schedules for this combination therapy (each “|” indicates a day of drug administration):

| | Days | | | | | | | |
|---|---|---|---|---|---|---|---|---|
| | Week | Week | Week | Week | Week | | | |
| | 1 | 2 | 3 | 4 | 5 | Week 6 | Week 7 | Week 8 |
| CAPE | \|\|\|\|\|\|\| | \|\|\|\|\|\|\| | \|\|\|\|\| | \|\|\|\|\| | \|\|\|\|\|\|\| | \|\|\|\|\|\|\| | \|\|\|\|\| | \|\|\|\|\| |
| ′573 | \|\|\|\|\| | \|\|\|\|\| | | | \|\|\|\|\| | \|\|\|\|\| | | |
| CAPE | \|\|\|\|\|\|\| | \|\|\|\|\|\|\| | \|\|\|\|\| | | \|\|\|\|\|\|\| | \|\|\|\|\|\|\| | \|\|\|\|\| | |
| tem | \|\|\|\|\| | | | | \|\|\|\|\| | | | |
| CAPE | \|\|\|\|\|\|\| | \|\|\|\|\|\|\| | \|\|\|\|\| | | \|\|\|\|\|\|\| | \|\|\|\|\|\|\| | \|\|\|\|\| | |
| ′573 | \|\|\|\|\| | | | | \|\|\|\|\| | | | |
| CAPE | \|\|\|\|\|\|\|\| | \|\|\|\|\|\|\|\| | \| | \| | \|\|\|\|\|\|\|\| | \|\|\|\|\|\|\|\| | \| | \| |
| ′573 | | | | | | | | |
| CAPE | \|\|\|\|\|\|\|\| | \|\|\|\|\|\|\|\| | \| | \| | \|\|\|\|\|\|\|\| | \|\|\|\|\|\|\|\| | \| | \| |
| tem | | | | | | | | |
| CAPE | \|\|\|\|\|\|\| | \|\|\|\|\|\|\| | \|\|\|\|\|\|\| | \|\|\|\|\|\|\| | \|\|\|\|\|\|\| | \|\|\|\|\|\|\| | \|\|\|\|\|\|\| | \|\|\|\|\|\|\| |
| ever | \|\|\|\|\|\|\| | \|\|\|\|\|\|\| | | | \|\|\|\|\|\|\| | \|\|\|\|\|\|\| | | |

Two 4-week cycles are shown above in which capecitabine (CAPE) is given daily for 14 days every 28 days, in combination, respectively, with AP23573 (′573) given p.o. QDx5, with temsirolimus (tem) or ′573 given by iv infusion QDx5 every other week, with tern or ′573 given i.v. once per week, or with everolimus (ever) given daily p.o., all at any of the dose levels disclosed in the box above or elsewhere herein.

The following depicts analogous schedules, but illustrating two 3-week cycles with administration of the capecitabine for 14-days every 21 days:

| | Days | | | | | |
|---|---|---|---|---|---|---|
| | Week 1 | Week 2 | Week 3 | Week 4 | Week 5 | Week 6 |
| CAPE | \|\|\|\|\|\|\| | \|\|\|\|\|\|\| | \|\|\|\|\| | \|\|\|\|\|\|\| | \|\|\|\|\|\|\| | \|\|\|\|\| |
| ′573 | \|\|\|\|\| | \|\|\|\|\| | | \|\|\|\|\| | \|\|\|\|\| | |
| CAPE | \|\|\|\|\|\|\| | \|\|\|\|\|\|\| | \|\|\|\|\| | \|\|\|\|\|\|\| | \|\|\|\|\|\|\| | |
| tem | \|\|\|\|\| | | | | \|\|\|\|\| | |
| CAPE | \|\|\|\|\|\|\| | \|\|\|\|\|\|\| | \|\|\|\|\| | \|\|\|\|\|\|\| | \|\|\|\|\|\|\| | |
| ′573 | \|\|\|\|\| | | | | \|\|\|\|\| | |
| CAPE | \|\|\|\|\|\|\|\| | \|\|\|\|\|\|\|\| | \| | \|\|\|\|\|\|\|\| | \|\|\|\|\|\|\|\| | \| |
| ′573 | | | | | | |
| CAPE | \|\|\|\|\|\|\|\| | \|\|\|\|\|\|\|\| | \| | \|\|\|\|\|\|\|\| | \|\|\|\|\|\|\|\| | \| |
| tem | | | | | | |
| CAPE | \|\|\|\|\|\|\| | \|\|\|\|\|\|\| | \|\|\|\|\|\|\| | \|\|\|\|\|\|\| | \|\|\|\|\|\|\| | \|\|\|\|\|\|\| |
| ever | \|\|\|\|\|\|\| | \|\|\|\|\|\|\| | | \|\|\|\|\|\|\| | \|\|\|\|\|\|\| | |

Again these are just a few, non-limiting examples illustrating, among other things, staggered co-administration.

This invention also provides a composition comprising an effective amount of the AP23573 (or other mTOR inhibitor) and capecitabine and at least one physiologically acceptable carrier or excipient. The Composition may be suitable for intravenous or oral administration. In various embodiments, the composition contains 2-50 mg of the mTOR inhibitor and 500-5000 mg, usually 500-1250 mg, of capecitabine and at least one physiologically acceptable carrier or excipient.

The composition may further comprise at least one additional therapeutic agent, for example, an additional chemotherapeutic agent.

This invention also provides a pharmaceutical kit comprising an mTOR inhibitor and capecitabine in one or more unit dosage forms for simultaneous, separate or sequential use in the treatment of a cancer in a patient. One or both of the drugs are formulated for i.v. administration, e.g., taking the form of one or more vials or pre-loaded syringes or other container holding a solution of either or both drugs, or one or more vials or other vessels containing lyophilized or concentrated drug packaged together with one or more containers of diluent, for example. Alternatively, one or both are formulated to be administered orally. Of greatest current interest are kits which contain capecitabine formulated for oral delivery and either AP23573, sirolimus or everolimus also formulated for oral delivery, or AP23573, sirolimus or temsirolimus formulated for iv delivery. Products formulated for oral administration, e.g., capsules, tablets, etc., may be packaged in blister packs, which can laid out and/or labeled in accordance with a selected dosing schedule. A wide variety of other packaging choices are of course available for practicing this aspect of the invention.

Given the documented activity of mTOR inhibitors against a wide variety of cancers, the combination therapy disclosed herein should be of interest for a correspondingly wide range of cancers. Those include among others prostate, endometrial, breast, ovarian, cervical, uterine, head and neck, small cell and non-small cell lung, pancreatic, kidney, brain, colorectal, bladder, mouth, larynx, esophagus and stomach cancers as well as various sarcomas (including the various bone and soft tissue sarcomas), melanomas, multiple myeloma, B-cell lymphoma, mantle cell lymphoma, Non-Hodgkin's Lymphoma, and leukemias such as ALL, CLL and CML, including, among others, cases which are advanced, recurrent, or refractory to one or more other therapies and/or metastatic.

Moreover, the discovery that an mTOR inhibitor can be administered in combination with capecitabine without unacceptable toxicity, as described above, opens the door for further combinations with additional drugs. Additional combinations of particular current interest involve the inclusion of a her2/EGFR inhibitor such as Tykerb (Laptinib), as an oral dose of 750-4500 mg/day, often 1000 or 1250 mg/day; Taxotere (docetaxel), administered by 60-minute or more i.v. infusion of 50-100 mg/m2, e.g., 75 mg/m2, once every three weeks; and Herceptin, given as a 90-minute i.v. infusion of 4 mg/kg followed by weekly iv infusions (may be 30-minute) of 2 mg/kg or as 6-8 mg/kg iv infusion once followed by 4-6 mg/kg doses once every three weeks. One or more of the foregoing agents may be included in the pharmaceutical methods, compositions and kits described above.

### BRIEF DESCRIPTION OF THE DRAWING

FIG. 1 depicts the administration schedule for the AP23573/capecitabine (CAPE) combination used in the Phase 1b Trial reported in Example 2. Also indicated are the time points for measuring pharmacodynamic (PD) and pharmacokinetic (PK) indicators (@=plasma levels of VEGF and PBMC levels of 4E-BP1, dihydropyrimidine dehydrogenase, thymidine phosphorylatse and thymidylate synthase. §=skin Bx (MAP kinase (MAPK), phosphoMAPK, phospho4E-BP1 . . . ).

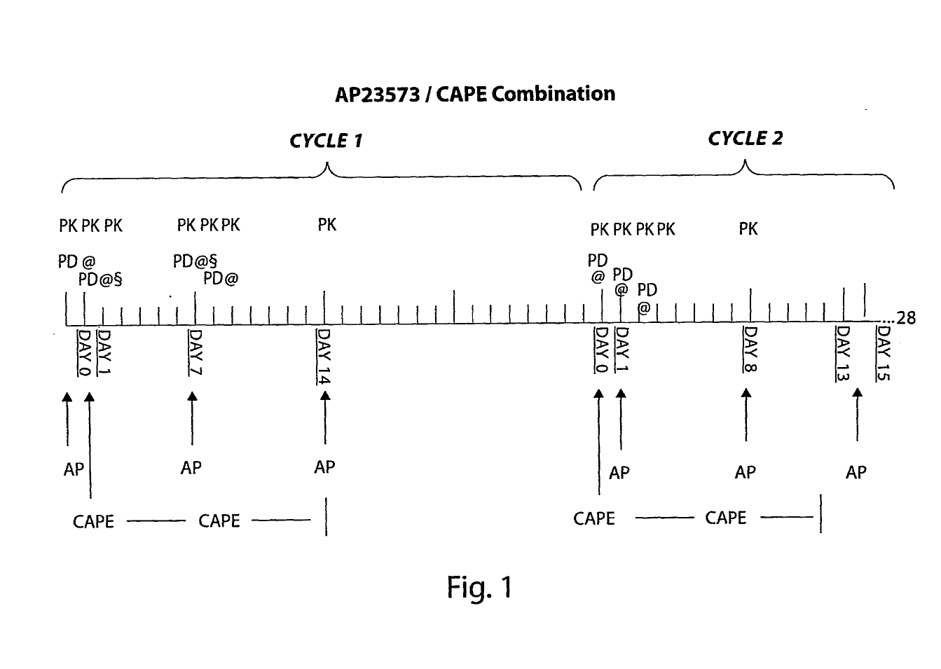

Table 1 shows characteristics of patients involved in the Phase 1b Trial described in Example 1.

FIG. 2 depicts the dose escalation strategy for the Phase 1b Trial described in Example 1.

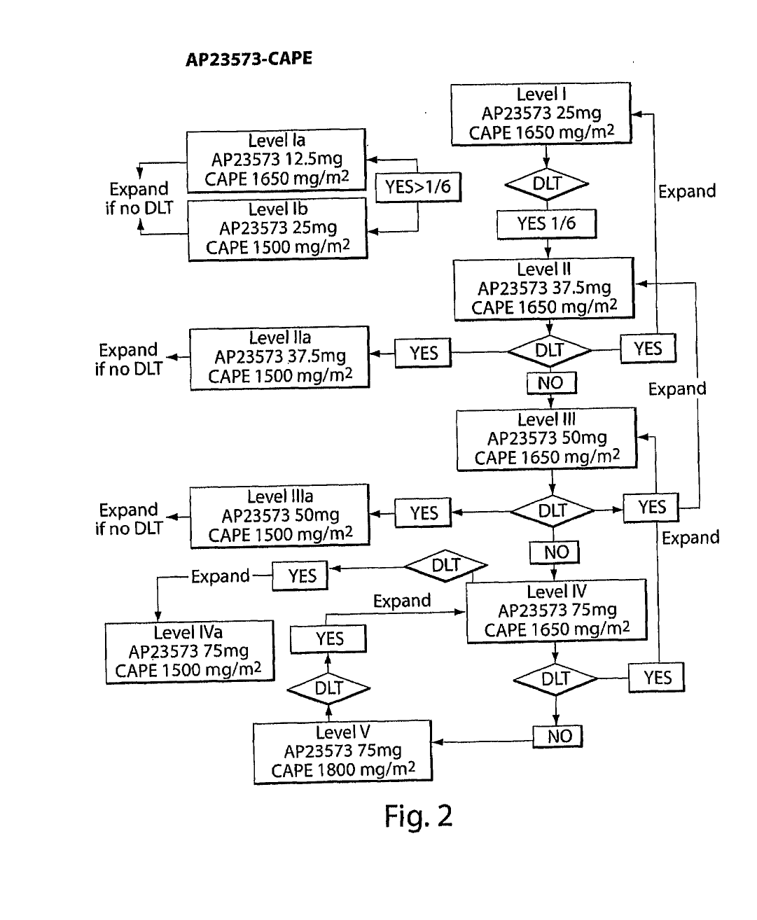

Table 2 shows a dose escalation summary for the first cycle's dose limiting toxicity cases (DLTS)

Table 3 shows the results of all cycle toxicity (% patient with drug related toxicity).

Table 4 shows results of tumor response.

FIG. 3 shows images taken from Patient 0005 (see Example 2 and Table 4) taken before treatment (image on the left), at cycle 2 (middle image), and 7 months after treatment (image on the right). Pt 005 (who received 3 cycles) is Off Study due to drug-related toxicity. Preliminary data suggests the patient remains in partial response (PR) during follow-up period.

FIG. 4 shows results of a pharmacokinetics study, where whole blood concentrations of AP23573 were measured by a HPLC MS/MS method. The graph shown on the left presents AP23573 profile after 30-minute infusion of 37.5 mg AP23573 co-administered with CAPE 1650 mg/m2/die with different sequences of administration (mean±SD of patients). The table, shown on the right, presents mean (±SD) pharmacokinetics parameters of AP23573 at two different doses.

FIG. 5 shows results of a pharmacokinetics study, where plasma levels of CAPE and its key metabolites (5-FU and 5FuH₂) were determined by HPLC analysis with UV detection at 310 nm for CAPE and at 205 nm for metabolites after using a liquid-liquid extraction method. The table presented in this figure shows Mean (±SD) pharmacokinetic parameters of CAPE (1650 mg/m²/die) and its key metabolites after the morning oral dose.

FIG. 6 shows results of the pharmacodynamic study, where DPD (dihydropyrimidine-dehydrogenase—graph on the left) and TP (thymidine phosphorilase—graph on the right) activity were measured in protein extracts prepared from PBMCs collected before and 24 hours after AP23573 on cycle 1 (Day 1 and day 8) and on cycle 2.

FIG. 7 is a graph showing representative mTOR inhibition in PBMCs following dosing with AP23573 and CAPE (see Example 2).

FIG. 8 is a graph presenting a summary of the preliminary results obtained on mTOR inhibition in PBMCs following dosing with AP23573 and CAPE.

### DEFINITIONS

Throughout the specification, several terms are employed that are defined in the following paragraphs.

The terms “subject”, “individual” and “patient” are used herein interchangeably. They refer to a human or another mammal (e.g., mouse, rat, rabbit, dog, cat, cattle, swine, sheep, horse or primate) that can be afflicted with or is susceptible to a disease or disorder (e.g., cancer) but may or may not have the disease or disorder. In many embodiments, the subject is a human being.

The term “treatment” as used herein means a method aimed at: (1) delaying or preventing the onset of a medical condition, disease or disorder; (2) slowing or stopping the progression, aggravation, or deterioration of the symptoms of the condition; (3) ameliorating the symptoms of the condition; and/or (4) curing the condition. The treatment may be administered prior to the onset of the condition, for a prophylactic or preventive action, or it may be administered after initiation of the condition, for a therapeutic action.

As used herein, the term “treatment effective amount”, or simply an “effective amount”, refers to any amount of a substance or composition that elicits a desired biological or medicinal response in a tissue, system or subject. For example, a desirable response may include one or more of the aims of treatment, as defined above.

As used herein, the term “co-administration” refers to administration of two or more biologically active substances to a subject. Co-administration can be simultaneous or sequential. The two or more biologically active substances can be part of a single composition or separate compositions.

A “pharmaceutical composition” is herein defined as comprising an amount of a drug, e.g., the mTOR inhibitor, capecitabine, etc., and at least one physiologically acceptable carrier or excipient. A pharmaceutical composition can further comprise various additional ingredients to aid or improve formulation, as well as one or more other therapeutic agents.

As used herein, the term “physiologically acceptable carrier or excipient” refers to a carrier medium or an excipient which does not interfere with the effectiveness of the biological activity of the active ingredient(s) of the composition and which is not excessively toxic to the host at the concentrations at which it is administered. The term includes diluents, bulking agents, anti-oxidants or other stabilizers, dispersants, solvents, dispersion media, coatings, antibacterial agents, isotonic agents, absorption delaying or enhancing agents, and the like. The use of such media and agents for the formulation of pharmaceutically active substances is well known in the art (see, for example, “Remington's Pharmaceutical Sciences”, E. W. Martin, 18^{th} Ed., 1990, Mack Publishing Co.: Easton, Pa., which is incorporated herein by reference in its entirety).

The terms “therapeutic agent” and “drug” are used herein interchangeably. They refer to a substance, molecule, compound, agent, factor or composition effective in the treatment of a disease or clinical condition.

As used herein, the term “chemotherapeutics” refers to those medications that are used to treat various forms of cancer. Anti-cancer drugs are conventionally classified in one of the following group: alkylating agents, anti-metabolite drugs, anti-mitotic antibiotics, alkaloidal anti-tumor agents, hormones and anti-hormones, interferons, non-steroidal anti-inflammatory drugs, and various other anti-tumor agents, including various antibodies and kinase inhibitors kinase inhibitors (e.g., inhibitors of Src, BRC/Abl, kdr, aurora-2, glycogen synthase kinase 3 or GSK-3, etc.). Anti-cancer drugs are generally given in a particular regimen over a period of weeks. Certain chemotherapeutic medications have the ability to directly kill cancer cells.

The term “cancer” refers to the physiological condition in mammals that is typically characterized by unregulated cell growth. Examples include, but are not limited to, carcinoma, lymphoma, blastoma, sarcoma, and leukemia. More particularly, examples of such cancers include squamous cell carcinoma, small-cell lung cancer, non-small cell lung cancer, pancreatic cancer, glioblastoma multiform, esophageal/oral cancer, cervical cancer, ovarian cancer, endometrial cancer, prostate cancer, bladder cancer, hepatoma, breast cancer, colon cancer, and head and neck cancer.

As used herein, the term “cancer patient” refers to an individual diagnosed with cancer (i.e., has actually tested positive for cancer) or to an individual suspected of having cancer (e.g., an individual that presents one or more symptoms indicative of cancer, has one or more risk factors, or is being screened for cancer). The term also includes individuals that have previously undergone therapy for cancer.

### DETAILED DESCRIPTION

As mentioned above, this invention provides methods and compositions involving the co-administration of capecitabine and an mTOR inhibitor for the treatment of cancer in mammals.

I—mTOR Inhibitors

mTOR inhibitors include any compound, or a pharmaceutically acceptable salt thereof, that inhibits cell replication by blocking the progression of the cell cycle from G1 to S phase. mTOR inhibitors of particular current interest include rapamycin (sirolimus) and analogs thereof that retain mTOR inhibitory activity, especially those noted elsewhere herein.

Rapamycin is a macrolide, discovered in the 1970's as a fermentation producr of Streptomyces hygroscopicus. Rapamycin is a potent immunosuppressive agent and is used clinically to prevent rejection of transplanted organs. It has also been reported to have a wide range of interesting pharmacologic activities, including certain anti-cancer activity. See e.g. US Pat. appln 2001/0010920.

Because there is more than one accepted convention for numbering the atoms of rapamycin and its analogs, the numbering convention used herein is depicted below:

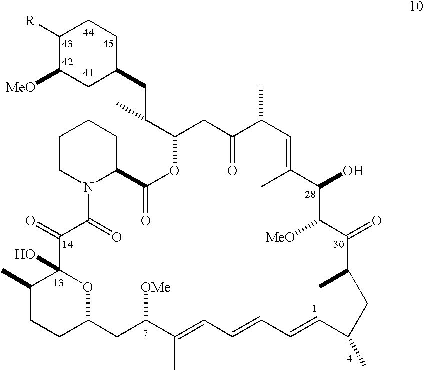

For reference, the R group for a number of compounds is set forth in the following table:

| Compound | —R |
|---|---|
| Rapamycin | —OH |
| AP23573 | —OP(O)(Me)₂ |
| Temsirolimus | —OC(O)C(CH₃)(CH₂OH) |
| Everolimus | —OCH₂CH₂OH |
| Biolimus | —OCH₂CH₂OEt |
| ABT-578 | -Tetrazole |

mTOR inhibitors currently in clinical development as anti-cancer agents include AP23573, temsirolimus and everolimus. After promising initial clinical studies, the potential clinical significance of these three compounds is being evaluated more fully in several phase II-III trials on patients with solid tumors and some hematological malignancies.

Temsirolimus is a soluble ester prodrug of rapamycin, rapamycin 42-ester with 3-hydroxy-2-(hydroxymethyl)-2-methylpropionic acid), which is disclosed in U.S. Pat. No. 5,362,718. CCI-779 has demonstrated significant inhibitory effects on tumor growth in both in vitro and in vivo models. CCI-779 exhibits cytostatic, as opposed to cytotoxic properties, and may delay the time to progression of tumors or time to tumor recurrence. As disclosed in WO 00/240000, CCI-779 may be useful for the treatment of cancers of various origins, including renal, breast, cervical, uterine, head and neck, lung, prostate, pancreatic, ovarian, colon, lymphoma and melanoma.

The mTOR inhibitor RAD001 (SDZ RAD, everolimus, Certican) is 40-0-(2-hydroxy)ethyl-rapamycin, the structure and synthesis of which is disclosed in WO 94/09010. RAD001, which has been shown to be a potent immunosuppressive agent (U.S. Pat. No. 5,665,772), also exhibits evidence of antineoplastic properties (see, e.g., A. Boulay et al., Cancer Res., 2004, 64: 252-261). As a result of these properties, RAD001 is currently marketed in certain countries as an immunosuppressant for prevention of allograft rejection (B. Nashan, Ther. Drug. Monit., 2002, 24: 53-58) and is undergoing clinical studies as an anti-cancer agent (S. Huang and P. J. Hougthon, Curr. Opin. Invest. Drugs, 2002, 3: 295-304; M. M. Mita et al., Clin. Breast Cancer, 2003, 4: 126-137; M. Hidalgo and E. J. Rowinsky, Oncogene, 2000, 19: 6680-6686).

The mTOR inhibitor of particular interest is AP23573, a phosphorous-containing rapamycin derivative (See WO 03/064383, Example 9 therein). Like CCI-779 and RAD001, AP23573 has demonstrated antiproliferative activity in a variety of PTEN-deficient tumor cell lines, including glioblastoma, prostate, breast, pancreas, lung and colon (E. K. Rowinsky, Curr. Opin. Oncol., 2004, 16: 564-575). AP23573 has been designated as a fast-track product by the U.S. Food and Drug Administration for the treatment of soft-tissue and bone sarcomas. AP23573 is currently in multiple clinical trials targeting hematologic malignancies (e.g., leukemias and lymphomas) and solid tumors (e.g., sarcomas, prostate cancer and gliobastoma multiforme).

These compounds are non-limiting examples of potent mTOR inhibitors. For additional information on AP23573, see U.S. Pat. No. 7,091,213. For recent references on temserolimus (CCI779), see WO 2004/026280, WO 2005/011688, WO 2005/070393, WO 2006/086172 and WO 2006/089312. For everolimus, see U.S. Pat. No. 6,384,046, U.S. Pat. No. 6,197,781, U.S. Pat. No. 6,004,973 and WO 2002/066019 and references cited therein. Other mTOR inhibitors of interest include 42-desmethoxy derivatives of rapamycin and its various analogs, as disclosed, e.g., in WO 2006/095185 (in which such compounds are referred to as “39-desmethoxy” compounds based on their numbering system). The derivatives of rapamycin are of particular current interest in practicing this invention

Additionally, a large number of other structural variants of rapamycin have now been reported, typically arising as alternative fermentation products and/or from synthetic efforts. For example, the extensive literature on analogs, homologs, derivatives and other compounds related stucturally to rapamycin (“rapalogs”) include, among others, variants of rapamycin having one or more of the following modifications relative to rapamycin: demethylation, elimination or replacement of the methoxy at C7, C42 and/or C29; elimination, derivatization or replacement of the hydroxy at C13, C43 and/or C28; reduction, elimination or derivatization of the ketone at C14, C24 and/or C30; replacement of the 6-membered pipecolate ring with a 5-membered prolyl ring; alternative substitution on the cyclohexyl ring or replacement of the cyclohexyl ring with a substituted cyclopentyl ring; epimerization of the C28 hydroxyl group; and substitution with phosphorous-containing moieties.

Thus, mTOR inhibitors include, for example, 43- and/or 28-esters, ethers, carbonates, carbamates, etc. of rapamycin including those described in the following patents, which are all hereby incorporated by reference: alkyl esters (U.S. Pat. No. 4,316,885); aminoalkyl esters (U.S. Pat. No. 4,650,803); fluorinated esters (U.S. Pat. No. 5,100,883); amide esters (U.S. Pat. No. 5,118,677); carbamate esters (U.S. Pat. No. 5,118,678); silyl esters (U.S. Pat. No. 5,120,842); aminodiesters (U.S. Pat. No. 5,162,333); sulfonate and sulfate esters (U.S. Pat. No. 5,177,203); esters (U.S. Pat. No. 5,221,670); alkoxyesters (U.S. Pat. No. 5,233,036); 0-aryl, -alkyl, -alkenyl, and -alkynyl ethers (U.S. Pat. No. 5,258,389); carbonate esters (U.S. Pat. No. 5,260,300); arylcarbonyl and alkoxycarbonyl carbamates (U.S. Pat. No. 5,262,423); carbamates (U.S. Pat. No. 5,302,584); hydroxyesters (U.S. Pat. No. 5,362,718); hindered esters (U.S. Pat. No. 5,385,908); heterocyclic esters (U.S. Pat. No. 5,385,909); gem-disubstituted esters (U.S. Pat. No. 5,385,910); amino alkanoic esters (U.S. Pat. No. 5,389,639); phosphorylcarbamate esters (U.S. Pat. No. 5,391,730); carbamate esters (U.S. Pat. No. 5,411,967); carbamate esters (U.S. Pat. No. 5,434,260); amidino carbamate esters (U.S. Pat. No. 5,463,048); carbamate esters (U.S. Pat. No. 5,480,988); carbamate esters (U.S. Pat. No. 5,480,989); carbamate esters (U.S. Pat. No. 5,489,680); hindered N-oxide esters (U.S. Pat. No. 5,491,231); biotin esters (U.S. Pat. No. 5,504,091); 0-alkyl ethers (U.S. Pat. No. 5,665,772); and PEG esters of rapamycin (U.S. Pat. No. 5,780,462). Also included are the reduced products, 24-dihydro-, 30-dihydro- and 24, 30-tetrahydro-rapamycin analogs and the 28-epi analogs (see, e.g., WO 01/14387) of rapamycin or of any of the foregoing compounds, as well as esters or ethers of any of the foregoing as well as oximes, hydrazones, and hydroxylamines of non-reduced compounds. See e.g. U.S. Pat. Nos. 5,373,014, 5,378,836, 5,023,264, 5,563,145 and 5,023,263.

Also of interest is ABT578, noted in the chart above, and the 43-epi isomer thereof, e.g., as disclosed in WO 99/15530, or rapamycin analogs as disclosed in No. WO 98/02441 and WO 05/016252.

Formulation of the mTOR Inhibitor:

A variety of oral and parenteral dosage forms are known for rapamycin and a number of rapamycin analogs. See e.g., U.S. Pat. No. 7,091,213. Some are currently in use in various treatment methods, monotherapies or otherwise. Those same dosage forms may likewise be used in the practice of the combination therapy disclosed herein. Solid dosage forms are often preferred for oral administration and include among others conventional admixtures, solid dispersions and nanoparticles, typically in tablet, capsule, caplet, gel cap or other solid or partially solid form. Such formulations may optionally contain an enteric coating. Numerous materials and methods for such oral formulations are well known. A typical example of the use of conventional materials and methods to formulate an mTOR inhibitor is shown in US Patent Application US 2004/0077677 and Published International Patent Application WO04026280 (CCI-779). See also U.S. Pat. No. 6,197,781, U.S. Pat. No. 6,589,536, U.S. Pat. No. 6,555,132, U.S. Pat. No. 5,985,321, U.S. Pat. No. 6,565,859 and U.S. Pat. No. 5,932,243.

In a preferred embodiment, the mTOR inhibitor is provided as an oral dosage form, such as a tablet. In the case of AP23573, for instance, the drug may prepared by a wet granulation process. The tablet may contain one or more cellulose polymers and one or more of an antioxidant, chelating agent, filler, binder, surfactant, desintegrant, lubricant, pH-modifying agent and the like. The wet granulation process may be performed with an aqueous or alcoholic, e.g., ethanol, solvent system. Other suitable alcohols include methanol, isopropanol, and the like. The solvent can also be a mixture of solvents, e.g. an alcoholic solvent and water.

It is currently of particular interest that the composition contain from 1 to 45%, from 2 to 35%, from 5 to 25%, or from 8 to 15% by weight of AP23573; from 1 to 50%, from 1 to 35%, from 1 to 15%, or from 2 to 15% by weight of cellulose polymer and from 0.01% to 3%, from 0.05% to 1% or from 0.05% to 0.5% by weight of antioxidant. However, various embodiments may contain more, or less, of these components.

Acceptable antioxidants include, but are not limited to, citric acid, d,l-α-tocopherol, BHA, BHT, monothioglycerol, ascorbic acid, and propyl gallate. It is expected that the antioxidants of the formulations of this invention will be used in concentrations ranging from 0.001% to 3% wt/wt.

Chelating agents, and other materials capable of binding metal ions, such as ethylene diamine tetra acetic acid (EDTA) and its salts are capable of enhancing the stability of AP23573.

Typical cellulose polymers include, but are not limited to hydroxypropylmethylcellulose (HPMC), hydroxypropylmethyl cellulose phthalate, methyl cellulose (MC), hydroxyethyl cellulose, and hydroxypropyl cellulose (HPC).

Acceptable pH modifying agents include, but are not limited to citric acid, sodium citrate, dilute HCl, and other mild acids or bases capable of buffering a solution containing AP23573 to a pH in the range of about 4 to about 6. If present in the composition, the pH modifying agent is usually in amount of up to 1%.

Surfactants may be present in the formulation and include polysorbate 80, sodium lauryl sulfate, sodium dodecyl sulfate, salts of bile acids (taurocholate, glycocholate, cholate, deoxycholate, etc.) which may be combined with lecithin. Alternatively, ethoxylated vegetable oils, such as Cremophor EL, vitamin E tocopherol propylene glycol succinate (Vitamin E TGPS), polyoxyethylene-polyoxypropylene block copolymers, and poloxamers. If present in the composition, the surfactant is usually in amount of up to 20%, for example 1 to 15% by weight.

Binders, fillers, and disintegrants such as sucrose, lactose, microcrystalline cellulose, croscarmellose sodium, magnesium stearate, gum acacia, cholesterol, tragacanth, stearic acid, gelatin, casein, lecithin (phosphatides), carboxymethylcellulose calcium, carboxymethylcellulose sodium, methyl cellulose, hydroxyethylcellulose, hydroxypropylcellulose, hydroxypropylmethyl-cellulose phthalate, noncrystalline cellulose, polyvinylpyrrolidone, cetostearyl alcohol, cetyl alcohol, cetyl esters wax, dextrates, dextrin, cyclodextrin, lactose, dextrose, glyceryl monooleate, glyceryl monostearate, glyceryl palmitostearate, polyoxyethylene alkyl ethers, polyethylene glycols, polyoxyethylene castor oil derivatives, polyoxyethylene stearates, and polyvinyl alcohol, and the like may also be incorporated into the formulation.

Any given formulation of this invention may contain multiple ingredients of each class of component. For example, a formulation containing an antioxidant may contain one or more antioxidants as the antioxidant component.

The tablet may further comprise a film-coat to control the release of the rapamycin analog. The tablet may be coated with a film-coat by spraying, dipping or by deposition. The film-coat typically includes a polymeric film-forming material such as copovidone (i.e a copolymer of polyvinylpyrrolidone and vinyl acetate), hydroxypropyl methylcellulose, hydroxypropylcellulose, and acrylate or methacrylate copolymers. Besides a film-forming polymer, the film-coat may further comprise a plasticizer, e.g. polyethylene glycol, triethyl citrate, a surfactant, e.g. a Tween™ type, an anti-foaming agent, e.g. Simethicone, and optionally a pigment, e.g. titanium dioxide or iron oxides. The film-coating may also comprise talc as anti-adhesive. The film coat usually accounts for less than about 5% by weight of the dosage form. In a preferred embodiment, the film-coating material comprises copovidone.

The film coating may also be an enteric layer comprising an enteric polymer, for delayed release of the rapamycin analog. An enteric layer is a coating of a substance (i.e a polymer) which is insoluble in the acid medium of the stomach but which is soluble at the higher pH encountered in the intestine. Such materials are used as film coatings on tablets to modify the release of a drug. Suitable enteric polymers are well known to those of skill in the art (WO 01/051031) and include, without limitation, methyl metacrylate polymers, methacrylic acid co-polymers, cellulose acetate phthalate, polyvinyacetate phthalate, hydroxypropyl methyl phthalate, and hydroxypropyl methyl cellulose phthalate. For instance, the enteric layer may comprise a methacrylic acid co-polymer such as Eudragit L100, Acryl-EZE or the like.

In addition to the foregoing non limiting examples of formulation technology, a wide variety of other methods and materials are also well known to those working in the field of macrolides like rapamycin and its derivatives. For additional background and examples of appropriate formulation technologies, see e.g., WO 03/064383 and US Published Patent Application 20050032825.

### II—Capecitabine

As previously noted, the pharmaceutical compositions and treatment methods of this invention involve the co-administration of an mTOR inhibitor with capecitabine.

Capecitabine (5′-deoxy-5-fluoro-N-[(pentyloxy)carbonyl]-cytidine (Xeloda™, Roche) is a fluoropyrimidine carbamate. It is currently approved by the US FDA for the treatment of metastatic colorectal cancer and metastatic breast cancer. It is also already approved in many other countries for the treatment of colorectal cancer. Readily absorbed by the gastrointestinal tract, capecitabine is metabolized by the enzyme carboxylesterase in the liver, where it is converted to 5′-deoxy-5-fluorocytidine (5′DFCR), which is then converted by the enzyme cytidine deaminase to 5′-deoxy-5-fluorouridine (5′ DFUR) (M. Miwa et al., Eur. J. Cancer, 1998, 34: 1274-1281; J. Schüller et al., Cancer Chemother. Pharmacol., 2000, 45: 291-297). In tumor and normal tissues, the enzyme thymidine phosphorylase (TP) converts 5′ DFUR to 5-fluorouracil (5-FU).

Unlike parenterally administered 5-FU, oral capecitabine concentrates predominantly in tumor tissue as opposed to adjacent healthy tissue and plasma (J. Schüller et al., Cancer Chemother. Pharmacol., 2000, 45: 291-297). As a result, orally administered capecitabine enables physicians treating breast cancer, for example, to mimic the effect of continuous 5-FU infusion but in a convenient outpatient setting without the complications and costs associated with infusion pumps and parenteral therapies (G. Liu et al., J. Clin. Oncol., 1997, 15: 110-115; M. Borner et al., Proc. Am. Soc. Clin. Oncol., 2000, 19: 191a).

Standard dosing with capecitabine as a monotherapy is 1,250 mg/m² orally twice daily (bid), morning and evening for 14 consecutive days in 3-week cycles.

### III—Other Anti-Cancer Agents

In certain embodiments, the mTOR inhibitor and capecitabine are co-administered along with one or more other chemotherapeutic drugs. Of greatest current interest are Tykerb™ (lapatinib), Taxotere™ (docetaxel) and Herceptin™ as previously described. The production, formulation and use of each of these is well known. Additionally, all three agents are commercially available.

Other anti-cancer drugs for use in conjunction with the combination described herein may be chosen from small molecules, peptides, saccharides, steroids, antibodies (including fragments or variants thereof), fusion proteins, antisense polynucleotides, ribozymes, small interfering RNAs, peptidomimetics, and the like, and include alkylating agents, alkaloidal anti-tumor agents, proteasome inhibitors and other inhibitors of NF-kB signaling, etc., although are preferably not additional anti-metabolite drugs.

Examples of chemotherapeutics include, but are not limited to, Zyloprim, alemtuzmab, altretamine, amifostine, nastrozole, antibodies against prostate-specific membrane antigen (such as MLN-591, MLN591RL and MLN2704), arsenic trioxide, Avastin™ (bevacizumab), (or other anti-VEGF antibody), bexarotene, bleomycin, busulfan, carboplatin, celecoxib, chlorambucil, cisplatin, cisplatin-epinephrine gel, cladribine, cytarabine liposomal, daunorubicin liposomal, daunorubicin, daunomycin, dexrazoxane, doxorubicin, Elliott's B Solution, epirubicin, estramustine, etoposide phosphate, etoposide, exemestane, gemtuzumab-ozogamicin, goserelin acetate, hydroxyurea, idarubicin, idarubicin, idamycin, ifosfamide, imatinib mesylate, irinotecan (or other topoisomerase inhibitor, including antibodies such as MLN576 (XR11576)), letrozole, leucovorin, leucovorin levamisole, liposomal daunorubicin, melphalan, L-PAM, mesna, methotrexate, methoxsalen, mitomycin C, mitoxantrone, MLN518 or MLN608 (or other inhibitors of the fit-3 receptor tyrosine kinase, PDFG-R or c-kit), itoxantrone, paclitaxel, Pegademase, pentostatin, porfimer sodium, Rituximab (RITUXAN™), talc, tamoxifen, temozolamide, teniposide, VM-26, topotecan, toremifene, an anti-Her2 antibody other than Herceptin, 2C4 (or other antibody which interferes with HER2-mediated signaling), tretinoin, ATRA, valrubicin, vinorelbine, or pamidronate, zoledronate or another bisphosphonates.

### IV—Other Therapeutic Agents

In certain embodiments, the mTOR inhibitor and capecitabine are co-administered according to the present invention with at least one additional therapeutic agent. Therapeutic agents suitable for such use include any drug whose administration may be beneficial to the subject receiving a composition of the present invention.

Thus, suitable therapeutic agents include:

Non Steroidal Anti-Inflammatory Drugs (NSAIDs), such as acetaminophen (Tylenol, Datril, etc), aspirin, ibuprofen (Motrin, Advil, Rufen, etc), choline magnesium salicylate (Triasate), choline salicylate (Anthropan), diclofenac (Voltaren, Cataflam, diflunisal (Dolobid), etodolac (Lodine), fenoprofen calcium (Naflon), flurobiprofen (Ansald), indomethacin (Indocin, Indometh, etc.), ketoprofen (Orudis, Oruvail), ketorolac tromethamine (Toradol), magnesium salicylate (Doan's, Magan, Mobidin, etc), meclofenamate sodium (Meclomen), mefenamic acid (Ponstel, Relafan), oxaprozin (Daypro), piroxicam (Feldene), sodium salicylate, sulindac (Clinoril), tolmetin (Tolectin), meloxicam (Mobic), nabumetone (Relafen), naproxen (Anaprox, Naprelan, Naprosyn, Aleve), lornoxicam, nimesulide (Nexen), indoprofen, salsalate (Disalcid, Salflex, etc), tiaprofenic acid (Surgam), flosulide, and the like;

analgesics/antipyretics (e.g., aspirin, acetaminophen, ibuprofen, naproxen sodium, buprenorphine hydrochloride, propoxyphene hydrochloride, propoxyphene napsylate, meperidine hydrochloride, hydromorphone hydrochloride, morphine sulfate, oxycodone hydrochloride, codeine phosphate, dihydrocodeine bitartrate, pentazocine hydrochloride, hydrocodone bitartrate, levorphanol tartrate, diflunisal, trolamine salicylate, nalbuphine hydrochloride, mefenamic acid, butorphanol tartrate, choline salicylate, butalbital, phenyltoloxamine citrate, diphenhydramine citrate, methotrimeprazine, cinnamedrine hydrochloride, meprobamate, and the like);

sedatives/hypnotics (e.g., barbiturates, such as pentobarbital, pentobarbital sodium, secobarbital sodium, benzodiazapines, such as flurazepam hydrochloride, triazolam, tomazeparm, midazolam hydrochloride, and the like);

antianginal agents (e.g., b-adrenergic blockers, calcium channel blockers, such as nifedipine, diltiazem hydrochloride, and the like, nitrates, such as nitroglycerin, isosorbide dinitrate, pentaerythritol tetranitrate, erythrityl tetranitrate, and the like);

antianxiety agents (e.g., lorazepam, buspirone hydrochloride, prazepam, chlordiazepoxide hydrochloride, oxazepam, clorazepate dipotassium, diazepam, hydroxyzine pamoate, hydroxyzine hydrochloride, alprazolam, droperidol, halazepam, chlormezanone, and the like);

antidepressants (e.g., doxepin hydrochloride, amoxapine, trazodone hydrochloride, amitriptyline hydrochloride, maprotiline hydrochloride, phenelzine sulfate, desipramine hydrochloride, nortriptyline hydrochloride, tranylcypromine sulfate, fluoxetine hydrochloride, doxepin hydrochloride, imipramine hydrochloride, imipramine pamoate, nortriptyline, amitriptyline hydrochloride, isocarboxazid, desipramine hydrochloride, trimipramine maleate, protriptyline hydrochloride, and the like);

antipsychotic agents (e.g., haloperidol, loxapine succinate, loxapine hydrochloride, thioridazine, thioridazine hydrochloride, thiothixene, fluphenazine hydrochloride, fluphenazine decanoate, fluphenazine enanthate, trifluoperazine hydrochloride, chlorpromazine hydrochloride, perphenazine, lithium citrate, prochlorperazine, and the like);

antimanic agents (e.g., lithium carbonate),

antiarrhythmics (e.g., bretylium tosylate, esmolol hydrochloride, verapamil hydrochloride, amiodarone, encainide hydrochloride, digoxin, digitoxin, mexiletine hydrochloride, disopyramide phosphate, procalnamide hydrochloride, quinidine sulfate, quinidine gluconate, quinidine polygalacturonate, flecalnide acetate, tocainide hydrochloride, lidocaine hydrochloride, and the like);

antihypertensive drugs, such as diuretics (hydrochlorothiazide, chlorthalidone, metolazone, indapamide, furosemide, bumetanide, torsemide, triamterene, amiloride, spronolactone), beta-adrenergic blocking agents (acebutolol, atenolol, betaxolol, cartelol, labetalol, metoprolol, nadolol, penbutolol, pindolol, propranolol, timolol), angiotensin converting enzyme inhibitors (benazepril, captopril, enalapril, fosinopril, quinoapril, ramimpril, losartan), calcium channel-blocking agents (diltiazem, verapamil, amlodipine, felodipine, isradipine, nicardipine, nifedipine), alpha-adrenoceptor blocking agents, sympatholytics, and vasodilators (such as prazosin, terazosin, doxazosin, clonidine, guanabenz, guanfacine, methylodopa, guanethidine, guanethidine monosulfate, reserpine, hydralazine, minoxidil, and the like), as well as agents such as trimethaphan camsylate, phenoxybenzamine hydrochloride, pargyline hydrochloride, deserpidine, diazoxide, rescinnamine, sodium nitroprusside, rauwolfia serpentina, alseroxylon, phentolamine mesylate, and the like;

antihistamine/antipruritic drugs, such as ethanolamines (e.g., diphenhydramine, diphenhydramine hydrochloride, clemastine, clemastine fumarate, and the like), ethylenediamines (e.g., brompheniramine, brompheniramine maleate, chlorpheniramine, chlorpheniramine maleate, dexchlorpheniramine maleate, triprolidine, triprolidine hydrochloride, and the like), phenothiazines (e.g., promethazine), piperidines (e.g., hydroxzine, hydroxyzine hydrochloride, terfenadine, astemizole, azatadine, azatadine maleate, and the like), cyproheptadine, cyproheptadine hydrochloride, loratidine, carbinoxamine maleate, diphenylpyraline hydrochloride, phenindamine tartrate, tripelennamine hydrochloride, methdilazine hydrochloride, trimprazine tartrate, and the like;

immunosuppressants, such as glucocorticoids (methylprednisolone), myelin basic protein (e.g., 7-capaxone), anti-Fc receptor monoclonal antibodies, hydroorotate dehydrogenase inhibitor, anti-IL2 monoclonal antibodies (e.g., CHI-621 and dacliximab), buspirone, castanospermine, CD-59 (complement factor inhibitor), 5-lipoxygenase inhibitor (e.g., CMI-392), phosphatidic acid synthesis antagonists, ebselen, edelfosine, enlimomab, galaptin, platelet activating factor antagonists, selectin antagonists (e.g., ICAM-4), interleukin-10 agonist, macrocylic lactone, methoxatone, mizoribine, OX-19, peptigen agents, PG-27, protein kinase C inhibitors, phosphodiesterase IV inhibitor, single chain antigen binding proteins, complement factor inhibitor, sialophorin, sirolimus, spirocyclic lactams, 5-hydroxytryptamine antagonist, anti-TCR monoclonal antibodies, CD5 gelonin and TOK-8801, and the like;

antimetabolite cytotoxics (azathioprine, cyclophosphamide), C5a release inhibitor, benzydamine, peldesine, pentostatin, SDZ-ASM-981, thalidomide, benzoporphyrin derivatives, arachidonate antagonists (e.g., halometasone, halobetasol propionate), corticosteriod (clobetasol propionate), growth hormone antagonists (octapeptide somatostatin analogue, lanreotide, angiopeptin and dermopeptin), thymopentin, and the like;

neuroprotective agents, such as a-adrenoreceptor antagonist (i.e., a-dihydroergocryptine), NMDA antagonists (e.g., 5,6,7-tichloro-THQTQ, remacemide, 2-piperazinecarboxylic acid, N-indologlycinamide derivatives, spiro[benzo(b)thiophen-4(5H) derivatives, CP-101606, eliprodil, dexanabinol, GV-150526, L-695902, L-701324, amantadine derivatives, dizocilpine, benzomorphan derivatives, aptiganel, (S)-a-phenyl-2-pyridine ethanamide dihydrochloride and 1-amino-cyclopentanecarboxylic acid), sodium channel antagonists (e.g., 619C89), glycine antagonists (e.g., glystasins), calcium channel antagonists (e.g., 3,5-pyridinedlcarboxylic acid derivatives, conopeptides, 1-piperazineethanol, thieno[2,3-b]pyridine-5-carboxylic acid derivatives, NS-3034, nilvadipine, nisoldipine, tirilazad mesylate, 2H-1-enzopyran-6-ol, nitrone spin traps, iacidipine, iomeerzine hydrochloride, lemildipine, lifarizine, CPC-304, efonidipine, F-0401, piperazine derivatives), calpain inhibitors, fibrinogen antagonists (e.g., ancrod), integrin antagonists (e.g., antegren), thromboxane A2 antagonist (e.g., 9H-carbazole-9-propanoic acid derivatives, 5-Heptenoic acid derivatives and 1-azulenesulfonic acid derivatives), brain-derived neurotropic factor, adrenergic transmitter uptake inhibitor (e.g., 1-butanamine), endothelin A receptor antagonists (e.g., benzenesulfonamide derivatives, GABA A receptor antagonists (e.g., triazolopyrimidine derivatives and cyclohexaneacetic acid derivatives), GPIIb IIIa receptor antagonists (e.g., C68-22), platelet aggregation antagonist (e.g., 2(1H)-quinolinone derivatives, 1H-pyrrole-1-acetic acid derivatives and coumadin), Factor Xa inhibitor, CPC-211, corticotropin releasing factor agonist, thrombin inhibitor (e.g., cothrombins, fraxiparine, dermatan sulfate and heparinoid), dotarizine, intracellular calcium chelators (e.g., BAPTA derivatives), radical formation antagonists (EPC-K1, 3-pyridinecarboxamide derivatives, superoxide dismutase, raxofelast, lubeluzole, 3H-pyrazol-3-one derivatives, kynurenic acid derivatives, homopiperazine derivatives, and polynitroxyl albumin), protein kinase inhibitors (e.g., 1H-1,4-diazepine), nerve growth agonist (e.g., floor plate factor-5), glutamate antagonist (e.g., cyclohexanepropanoic acid, riluzole, NS-409 and acetamide derivatives), lipid peroxidase inhibitor (e.g., 2,5-cyclohexadiene-1,4-dione derivatives), sigma receptor agonist (e.g., cyclopropanemethanamine derivatives and SA-4503), thyrotropin releasing hormone agonist (e.g., JTP-2942, L-prolinamide and posatirelin), prolyl endopeptidase inhibitor, monosialogangiloside GM1, proteolytic enzyme inhibitor (e.g., nafamostat), neutrophil inhibitory factor, platelet activating factor antagonist (e.g., nupafant), monoamine oxidase B inhibitor (e.g., parafluoroselegiline and benzonitrile derivatives), PARS inhibitors, Angiotensin I converting enzyme inhibitor (e.g., perindopril and ramipril), acetylcholine agonist (e.g., pramiracetam), protein systhesis antagonist (e.g., procysteine), phosphodiesterase inhibitor (e.g., propentofylline), opioid kappa receptor agonist (e.g., 10H-phenothiazine-2-carboxamine derivatives), complement factor inhibitor (sCRI fragments), somatomedin-1, carnitine acetyltransferase stimulant (e.g., acetylcarnitine), and the like;

T cell inhibitors such as synthetic leucocyte antigen derived peptides, interleukin-1 receptor antagonist, MG/AnergiX, anti-CD3 monoclonal antibodies, anti-CD23 monoclonal antibodies, anti-CD28 antibodies, anti-CD2 monoclonal antibodies, CD4 antagonists, anti-E selectin antibodies, MHC inhibitors, monogens, mycophenolate mofetil, LRA-1 inhibitors, selectin inhibitors, and the like;

antimigraine agents, such as MK-462, 324C91, Phytomedicine, (S)-fluoxetine, calcium channel antagonists (e.g., nimodipine/Nimotop, flunarizine, dotarizine/FI-6026, iomerizine HCL/KB-2796, CPC-304, and CPC-317), a-dihydroergocryptine, 5-HT1 agonists, (e.g., Sumatriptan/Imitrex, Imigran, GR-85548, 311C, and GR-127607), 5-HT1D agonists, 5-HT1A antagonists, 5-HT1B antagonists (e.g., CP-93129), 5-HT1D antagonists (e.g., 1H-indole-5-ethanesulfonamide derivatives and 1H-indole-5-methanesulfonamide), 5-HT1D receptor cloned (e.g., 5-HT1D agents), 2-thiophenecarboxamide, 3-piperidinamine, diclofenac potassium, dihydroergotamine (e.g., DHE 45), ergotamine tartrate, dolasetron mesilate, dotarizine, flupirtine, histamine-H3 receptor agonist, indobufen, 1-azulenesulfonic acid derivatives, cholinesterase inhibitors, (e.g., S-9977), bradykinin antagonists, nitric oxide reductase inhibitors (e.g., BN-52296), nitric oxide receptor antagonists, substance P antagonists (e.g., Capsaicin/Nasocap), endopeptidase inhibitors (e.g., neutral endopeptidase, cloned), piperazine derivatives, neurokinin 1 antagonists, metergoline, dopamine D2 antagonist (e.g., metoclopramide +lysine acetyl), enkephalinase inhibitors (e.g., neutral endopeptidase), 5-HT2 antagonists (e.g., LY-053857), 5-HT3 antagonists (e.g., Dolasetron mesilate/MDL-73147, and 4H-carbazol-4-one derivatives), tenosal, tolfenamic acid, cyclooxygenase inhibitors (e.g., carbasalate/carbaspirin calcium, and tenosal/MR-Y134), alpha adrenoreceptor antagonists (e.g., arotinolol, and dihydroergocryptine), opioid agonists (e.g., flupirtine/D-9998), beta adrenergic antagonists (e.g., propranolol), valproate semisodium, propanolol hydrochloride, isometheptene mucate, dichloralphenazone, and the like;

antigout agents (e.g., colchicine, allopurinol, and the like);

anticoagulants (e.g., heparin, heparin sodium, warfarin sodium, and the like);

thrombolytic agents (e.g., urokinase, streptokinase, altoplase, and the like);

antifibrinolytic agents (e.g., aminocaproic acid);

hemorheologic agents (e.g., pentoxifylline and the like);

antiplatelet agents (e.g., aspirin, empirin, ascriptin, and the like);

anticonvulsants (e.g., valproic acid, divalproate sodium, phenytoin, phenytoin sodium, clonazepam, primidone, phenobarbitol, phenobarbitol sodium, carbamazepine, amobarbital sodium, methsuximide, metharbital, mephobarbital, mephenytoin, phensuximide, paramethadione, ethotoin, phenacemide, secobarbitol sodium, clorazepate dipotassium, trimethadione, and the like);

agents useful for calcium regulation (e.g., calcitonin, parathyroid hormone, and the like);

antibacterial agents (e.g., amikacin sulfate, aztreonam, chloramphenicol, chloramphenicol palmitate, chloramphenicol sodium succinate, ciprofloxacin hydrochloride, clindamycin hydrochloride, clindamycin palmitate, clindamycin phosphate, metronidazole, metronidazole hydrochloride, gentamicin sulfate, lincomycin hydrochloride, tobramycin sulfate, vancomycin hydrochloride, polymyxin B sulfate, colistimethate sodium, colistin sulfate, and the like);

antifungal agents (e.g., griseofulvin, keloconazole, and the like);

antiviral agents (e.g., interferon g, zidovudine, amantadine hydrochloride, ribavirin, acyclovir, and the like);

antimicrobials (e.g., cephalosporins, such as cefazolin sodium, cephradine, cefaclor, cephapirin sodium, ceftizoxime sodium, cefoperazone sodium, cefotetan disodium, cefutoxime azotil, cefotaxime sodium, cefadroxil monohydrate, ceftazidime, cephalexin, cephalothin sodium, cephalexin hydrochloride monohydrate, cefamandole nafate, cefoxitin sodium, cefonicid sodium, ceforanide, ceftriaxone sodium, ceftazidime, cefadroxil, cephradine, cefuroxime sodium, and the like; penicillins, such as ampicillin, amoxicillin, penicillin G benzathine, cyclacillin, ampicillin sodium, penicillin G potassium, penicillin V potassium, piperacillin sodium, oxacillin sodium, bacampicillin hydrochloride, cloxacillin sodium, ticarcillin disodium, aziocillin sodium, carbenicillin indanyl sodium, penicillin G potassium, penicillin G procaine, methicillin sodium, nafcillin sodium, and the like; erythromycins, such as erythromycin ethylsuccinate, erythromycin, erythromycin estolate, erythromycin lactoblonate, erythromycin siearate, erythromycin ethylsuccinate, and the like; tetracyclines, such as tetracycline hydrochloride, doxycycline hyclate, minocycline hydrochloride, and the like; etc);

antioxidants (e.g., N-acetylcsysteine, Vitamin A, Vitamin C, Vitamin E, b-carotene, EUK-8, flavonoids, glutathione, a-lipoic acid, melatonin, retinols, and the like);

anti-infectives (e.g., miconazole, vidarabine, inosine, pranobex, vidarabine, inosine prabonex, cefpimizole sodium), fradiomycin, and the like);

bronchodialators (e.g., sympathomimetics, such as epinephrine hydrochloride, metaproterenol sulfate, terbutaline sulfate, isoetharine, isoetharine mesylate, isoetharine hydrochloride, albuterol sulfate, albuterol, bitolterol, mesylate isoproterenol hydrochloride, terbutaline sulfate, epinephrine bitartrate, metaproterenol sulfate, epinephrine, epinephrine bitartrate; anticholinergic agents, such as ipratropium bromide; xanthines, such as aminophylline, dyphylline, metaproterenol sulfate, aminophylline; mast cell stabilizers, such as cromolyn sodium; inhalant corticosteroids, such as flurisolidebeclomethasone diproplonate, beclomethasone dipropionate monohydrate; salbutamol; beclomethasone dipropionate (BDP); ipratropium bromide; budesonide; ketotifen; salmeterol; xinafoate; terbutaline sulfate; triamcinolone; theophylline; nedocromil sodium; metaproterenol sulfate; albuterol; flunisolide) and the like;

hormones (e.g., androgens, such as danazol, testosterone cypionate, fluoxymesterone, ethyltostosterone, testosterone enanihate, methyltestosterone, fluoxymesterone, testosterone cypionate; estrogens, such as estradiol, estropipate, conjugated estrogens; progestins, such as methoxyprogesterone acetate, norethindrone acetate; corticosteroids, such as triamcinolone, betamethasone, betamethasone sodium phosphate, dexamethasone, dexamethasone sodium phosphate, dexamethasone acetate, prednisone, methylprednisolone acetate suspension, triamcinolone acetonide, methylprednisolone, prednisolone sodium phosphate methylprednisolone sodium succinate, hydrocortisone sodium succinate, methylprednisolone sodium succinate, triamcinolone hexacatonide, hydrocortisone, hydrocortisone cypionate, prednisolone, fluorocortisone acetate, paramethasone acetate, prednisolone tebulate, prednisolone acetate, prednisolone sodium phosphate, hydrocortisone sodium succinate, and the like; thyroid hormones, such as levothyroxine sodium, and the like;

hypoglycemic agents (e.g., human insulin, purified beef insulin, purified pork insulin, glyburide, chlorpropamide, glipizide, tolbutamide, tolazamide, and the like);

hypolipidemic agents (e.g., clofibrate, dextrothyroxine sodium, probucol, lovastatin, niacin, and the like);

proteins (e.g., DNase, alginase, superoxide dismutase, lipase, and the like);

nucleic acids (e.g., sense or anti-sense nucleic acids encoding any therapeutically active protein, including the proteins described herein, and the like);

agents useful for erythropoiesis stimulation (e.g., erythropoietin);

antiulcer/antireflux agents (e.g., famotidine, cimetidine, ranitidine hydrochloride, and the like);

antinauseants/antiemetics (e.g., meclizine hydrochloride, nabilone, prochlorperazine, dimenhydrinate, promethazine hydrochloride, thiethylperazine, scopolamine, and the like);

septic shock agents, anti-inflamatory agents and angiogenesis inhibitors (OLX-514), bradykinin antagonists (e.g., CP-0502, and NPC-1773 1), complement factor inhibitors (e.g., C3 convertase inhibitor), C5a release inhibitors (e.g., CAB-2.1), dopamine agonists (e.g., dopexamine), elastase inhibitors (e.g., ONO-5046), E selectin antagonists (e.g., CY-1787), farnesyltransferase inhibitors (RBE limonene), immunostimulants (e.g., CGP-19835A, lipid A vaccine, edobacomab, nebacumab, StaphGAM, and diabodies), immunosuppressants (e.g., CytoTAB, and transcyclopentanyl purine analogues), interleukin 1 antagonists (e.g., interleukin 1 receptors), interleukin 1 receptor antagonists (e.g., anakinra), interleukin 1b antagonists (e.g., interleukin-1b), interleukin 1b converting enzyme inhibitors (e.g., ICE-inhibitors), interleukin 8 antagonists (e.g., IL-8 receptor), interleukin 13 agonists (e.g., intereleukin-13), ITF-1697, lipase clearing factor inhibitors (e.g., SC-59735), membrane permeability enhancers (e.g., Bactericidal Permeability Increasing protein/BPI), nitric oxide antagonists (e.g., hydroxocobalamin), nitric oxide synthase inhibitors (e.g., L-NMMA, and a-methyl-N-delta-iminoethyl-ornithine), P2 receptor stimulants (e.g., ATP analogues), phosphatidic acid synthesis antagonists (e.g., lisofylline), phospholipase A2 inhibitors (e.g., S-448, acylpyrrole-alkanoic acid derivatives, and indoleacetic acid derivatives), platelet activating factor antagonists (e.g., ABT-299, TCV-309, SM-12502, (2RS,4R)-3-(2-(3-pyridinyl)-thiazolidin-4-oyl)indoles, UR-12670, and E-5880), prostacyclin agonists (e.g., taprostene), prostaglandin E1 agonists (e.g., TLC C-53), protein kinase inhibitors (e.g., SB-203580), protein kinase C inhibitors, protein synthesis antagonists (e.g., procysteine), proteolytic enzyme inhibitors (e.g., nafamostat), SDZ-PMX-622, selectin antagonists (e.g., sulfated glycolipid cell adhesion inhibitors), thrombin inhibitors (e.g., GS-522), TNF receptor-Ig, tumor necrosis factor antagonists (e.g., anti-TNF MAbs, MAK-195F, TBP-I, Yeda, rhTNFbp, and CDP-571), tumor necrosis factor alpha antagonists (e.g., E-5531), and the like;

multiple sclerosis agents, such as 4-aminopyridine, 15±deoxyspergualin, ACTH, amantadine, antibody adjuvants (e.g., poly-ICLC, and poly-IC+poly-L-lysine+carboxymethylcellulose), anti-cytokine MAb (CDP-835), anti-inflammatory (e.g., CY-1787, and CY-1503), anti-selectin MAb (e.g., CY-1787), anti-TCR MAb (e.g., NBI-114, NBI-115, and NBI-116), bacloten, bethanechol chloride, carbamazepine, carbohydrate drugs (e.g., CY-1503), clonazepam, CNS and immune system function modulators (e.g., NBI-106, and NBI-107), cyclophosphamide, cyclosporine A, cytokines (e.g., IFN-a, alfaferone, IFN-b, betaseron, TGF-b, PEG-TGF-b, betakine, IFN-b/Rebif, frone, interferon-b, and IFN-b), CD4+T cell inhibitors (e.g., AnergiX), CD28 antagonists (e.g., B7-1, B7-2, and CD28), direct cytotoxicity therapies (e.g., benzoporphyrin derivative (BPD)), FK-506, growth factors (e.g., glial growth factor, GGF, nerve growth factors, TGF-b, PEG-TGF-b, and betakine), humanized MAb (e.g., anti-IFN-g MAb, smart anti-IFN-g MAb, anti-Tac antibody, and smart anti-Tac antibody), humanized anti-CD4 MAb (e.g., anti-CD4 MAb, centara), hydrolase stimulants (e.g., castanospermine), IFN-a., IFN-g antagonist (e.g., anti-IFN-g MAb, and smart anti-IFN-g MAb), IL-2 antagonists (e.g., tacrolimus, FK-506, FR-900506, Fujimycin, Prograf, IL-2 fusion toxin, and DAB389 IL-2), IL-4 antagonists (e.g., IL-4 fusion toxin, and DAB389 IL-4), immune-mediated neuronal damage inhibitors (e.g., NBI-114, NBI-115, and NBI-116), immunoglobins, immunostimulants (e.g., poly-ICLC, edelfosine, ALP, ET-18-OCH3, ET-18-OME, NSC-24, and poly-IC+poly-L-lysine+carboxymethyl-cellulose), immunosuppressants (e.g., azathioprine, Al-100 animal protein, rDNA human protein AI-101, peptide, AI-102, castanospermine, tacrolimus, FK-506, FR-900506, Fujimycin, Prograf, anti-leukointegrin MAb, Hu23F2G, primatized anti-CD4 antibody, CE9.1, Galaptin 14-1, GL14-1, Lectin-1, recombinant IML-1, linomide, roquinimex, LS-2616, transcyclo-pentanyl purine analogs, MS-6044, spanidin, 15-deoxyspergualin, deoxyspurgiline, gusperimus HCL, NSC-356894, NKT-01, TCR, CD3/Ti, cyclosporine, OL-27-400, Sandimmune, Human IL-10, monogens, anti-TCR MAbs, TCAR MAbs, Monogen TM19, Monogen TM27, Monogen TM29, Monogen TM31, peptigen TP12 anti-CD4 MAb, cantara, immunophilins, VX-10367, VX-10393, VX-10428, synthetic basic copolymer of amino acids, copolymer-1, COP-1, T lymphocyte immunofusion (TIF) protein, and cyclophosphamide), integrin antagonists (e.g., anti-integrln (cell adhesion molecule a4 integrin) MAbs, AN-100225, and AN-100226), interferon agonists (e.g., poly-ICLC, and poly-IC+poly-L-lysine+carboxymethyl-cellulose), interferon-b-1b, isoprinosine, IV methylprednisolone, macrolides (e.g., tacrolimus, FK-506, FR-900506, Fujimycin, and Prograf), MAO B inhibitors (e.g., selegiline, and Parkinyl), methotrexate, mitoxantrone, muscle relaxants (e.g., RGH-5002), muscarinic antagonists (e.g., RGH-5002), neurosteroids (e.g., NBI-106, and NBI-107), octapeptides (e.g., peptide T), oxybutinin chloride, oxygen free radical antagonists (e.g., tetrandrine, biobenzylisoquinoline alkaloid), peptide agonists (e.g., peptide T), phenoxybenzamine, phospholipase C inhibitors (e.g., edelfosine, ALP, ET-18-OCH3, ET-18-OME, NSC-24), photodynamic therapies (e.g., benzoporphyrin derivative (BPD)), plasmapheresis, platelet activating factor antagonists (e.g., ginkgolide B, and BN-52021), potassium channel antagonists (e.g., aminodiaquine, and EL-970), propranolol, prostaglandin synthase inhibitors (e.g., sulfasalazine, salazosulfa-pyridine, PJ-306, SI-88, azulfidine, salazopyrin), protease antagonists (e.g., ginkgolide B, and BN-52021), recombinant soluble IL-1 receptors, spergualin analogs (e.g., spanidin, 15-deoxyspergualin, deoxyspurgiline, gusperimus HCl, NSC-356894, NKT-01), TCR peptide decoys (e.g., NBI-114, NBI-115, and NBI-116), TCR peptidomimetic decoys (e.g., NBI-114, NBI-115, and NBI-116), TCR peptide vaccines (e.g., AI-208), selectin antagonists (e.g., lectin-1, and recombinant IML-1), soluble TNF receptor I, TCARs (e.g., TCR, CD3/Ti, and peptigen TP12), TNF antagonists (e.g., thalidomide, and TNF inhibitors), tricyclic antidepressants, and the like;

organ transplantation agents, such as anti-CD25 MAbs, anti-Tac antibodies, anti-TNF MAb (e.g., CDP571), apoptosin, azathioprines (e.g., imuran), BCX-34, CA3, CD28, complement inhibiting factors (e.g., CD59), CTLA4Ig, cyclosporines (e.g., CsA), FK-506/rapamycin binding proteins (FKBP), glucocorticoids, humanized version of OKT3 (e.g., huOKT3-185), mycophenolate mofetil, hydroorotate dehydrogenase inhibitors (e.g., Brequinar), orthoclone OKT3 (e.g., IgG2a anti-T cell murine monoclonal antibody, and muromonab-CD3), and streptomyces isolates (e.g., FR-900520, and FR-900523), and the like;

systemic lupus erythematosus (SLE) agents, such as androgen-derived steriods (e.g., Org-4094), anti-CD4 humanized antibodies, anti-DNA/V-88, anti-idiotypic murine MAb (e.g., anti-idiotypic antibody to 3E10/MAb1 C7), CD2 antagonists (e.g., CD2), complement inhibitors (e.g., recombinant MCP-based complement inhibitors), cyclosporines (e.g., Sandimmune, cyclosporine analog, OG-37325, cyclosporin-G, and NVaI-CyA), cytokines (e.g., IL-4 fusion toxin), cytokine receptor antagonists (e.g., immunomodulatory cytokines), E-selectin antagonists (e.g., anti-ELAM, and CY-1787), FK506/tacrolimus (e.g., Prograf), hypercalcemic agents (e.g., KH-1060), IFN-g antagonists (e.g., anti-IFN-g MAb, and smart anti-IFN-g MAb), IL-1b converting enzyme inhibitors (ICE), IL-2 produced by E. coil (e.g., celmoleukin, IL-2, TGP-3, and Celeuk), immunoglobulins (e.g., anti-ELAM, CY-1788, and humanized CY-1787), immunostimulants (e.g., thymotrinan, RGH-0205, and TP3), immunosuppressants (e.g., Rapamycin, AY-22989, NSC-226080, NSC-606698, anti-CD4, T-cell inhibitor, anti-tac MAb, smart anti-tac MAb, Migis (membrane immunoglobulin-isotope specific) antibodies, SM-8849, immunophilins, VX-10367, VX-10393, VX-10428, mycophenolate mofetil, ME-MPA, RS-61444, cyclosporine, OL-27-400, Sandimmune, IL-4 fusion toxin, trypanosomal inhibitory factor (TIF), T-cell receptor, CD3/Ti, Org-4094, anti-TBM, CP 17193, Leflunomide/A-77-1726, ELAM-1, AnergiX, Spanidin, 15-deoxyspergualin, deoxyspurgiline, gusperimus hydrochloride, NSC-356894, NKT-01, Roquinimex, LS-2616, linomide, LJP-394, and CD-59 antigen), immunotoxins (e.g., Zolimomab aritox, xmmly-h65-rta, xomazyme-lym/CD5-Plus, OrthoZyme-CD5+, XomaZyme-H65-rta, Xomazyme-CD5 Plus), intravenous immunoglobulins (e.g., IVIG), integrin antagonists (e.g., integrin blockers), Migis™ antibodies, monoclonal antibody therapeutics, murine MAb (e.g., anti-SLE vaccine, and MAb 3E10), primatized anti-CD4 antibodies (e.g., CE9.1), protease inhibitors (e.g., matrix metalloprotease (MMP) inhibitors, and stromelysin), protein synthesis antagonists (e.g., anti-CD6-bR, anti-T12-bR, and oncolysin CD6), purine nucleoside phosphorylase inhibitors (e.g., BCX-25, and BCX-14), selectin antagonists (e.g., CY1503, and Cylexin), spergualin analogues (e.g., Spanidin, 15-deoxyspergualin, deoxyspurgiline, gusperimus hydrochloride, NSC-356894, and NKT-01), T cell inhibitors (e.g., AnergiX), tumor necrosis factor (TNF) antagonists, and the like;

Alzheimer's disease agents, such as ACh release enhancers (e.g., T-588 (benzothiophene derivative)), acetylcholine release stimulants (e.g., DUP-996 and analogues), AMPA agonists (e.g., AMAlex, and isoxazole compound series), AMPA GluR agonist (e.g., IDRA-21 [7-chloro-3-methyl-3,4-dihydro-2H-1,2,4-benzothiadiazinine]), AMPA GluR antagonists (e.g., S-18986, and related quinolone derivatives), anticholinesterases (e.g., E-2020), Ca-antagonists (e.g., NS-649, spider venom-derived ICM peptides and analogues, and substituted 2-aminoindanes compound series), combined anticholinesterase and muscarinic AChR antagonists (e.g., PD 142676), K-channel blockers (e.g., Trans-R-4-(4-methoxyphenyl-methyl)cyclohexylanine and analogues, and margatoxin-based functional and/or structural analogues), MI muscarinic receptor agonists (e.g., Xanomeline), NMDA antagonists (e.g., certain indole derivatives, and (R—(R1,S 1))-.alpha.-(4-hydroxyphenyl)-beta-methyl-4-(phenylmenthyl)-1-piperidinepropanol and analogues), nicotinic AChR agonists (e.g., ABT-418 [isoxazole, 3-meth-5-(1-meth-2-pyrrolidinyl)]), and the like;

antiparkinson agents (e.g., ethosuximide, and the like);

other agents such as psoriasis agents, such as 5-LO inhibitors (e.g., Wy-50295,Wy-49232, Lonapalene, RS-43179, MK-886, L-663536, EFH-615, DUP-654, Zileuton, epocarbazolin-A, and A-64077), 5-LO/CO inhibitors (e.g., BF-397, Tenidap, CP-309, and CP-66248), angiogenesis inhibitors (e.g., platelet factor 4), anticancer antibiotic (e.g., AGM-1470, and TNP-470), anti-inflammatory cytochrome P450 oxidoreductase inhibitors (e.g., DuP-630, and DuP-983), antiproliferative compounds (e.g., Zyn-Linker), arachidonic acid analogues (e.g., CD581, and CD554), arachidonic acid antagonists (e.g., Lonopalene, RS-43179, triamcinolone acetonide with penetration enhancer Azone, betamethasone dipropionate steroid wipe, G-202, Halobetasol propionate, ultravate, Halometasone, C-48401-Ba, and Sicorten), beta-glucan receptor antagonists, betamethasone steroid wipes, calcium metabolic moderators (e.g., Tacalcitol, Bonealfa, TV-02 ointment, Ro-23-6474, KH-1060, Calcipotriol, BMS-181161, BMY-30434, Dovonex, and Divonex), CD4 binding inhibitors (e.g., PIC 060), cell adhesion compounds (e.g., CY-726, VCAM-1, ELAM-1, and ICAM), cell adhesion inhibitors (e.g., selectin inhibitor, GM-1930), cellular aging inhibitors (e.g., Factor X), corticosteroids (e.g., Halobetasol propionate, ultravate, Halometasone, C-48401-Ba, and Sicorten), cyclosporin analogues (e.g., IMM-125), dihydrofolate reductase inhibitors (e.g., G-301, dichlorobenzoprim, methotrexate, and methotrexate in microsponge delivery system), E-selectin inhibitors (e.g., ISIS 4730), endogenous active form of vitamin D3 (e.g., Calcitriol, and Du-026325), fibroblast growth factor antagonists (e.g., Saporin mitotoxin, and Steno-Stat), fumagillin analogues (e.g., AGM-1470, and TNP-470), G-proteins and signal transduction compounds (e.g., CPC-A), gel formulations for acne (e.g., nicotinamide, N-547, and Papulex), growth hormone antagonists (e.g., Octreotide, Sandostatin, Lanreotide, angiopeptin, BIM-23014, and Somatuline), humanized antibodies (e.g., anti-CD4 antibody), hydroorotate dehydrogenase inhibitors (e.g., Brequinar sodium, bipenquinate, and DuP-785), ICAM-1 inhibitors (e.g., ISIS 939), IL-1 and other cytokine inhibitors (e.g., Septanil), IL-1 converting ezyme inhibitors, IL-1 receptor antagonists (e.g., Antril), IL-2 antagonists (e.g., Tacrolimus, Prograf, and FK-506), IL-2 receptor-targeted fusion toxins (DAB389IL-2), IL-8 receptors, immunostimulants (e.g., Thymopentin, and Timunox), immunosuppressants (e.g., XomaZyme-CD5 Plus, cyclosporine, Sandimmune, SR-31747, anti-CD 11, 18 MAb, Tacrolimus, Prograf, FK-506, and FK-507), immunosuppressive agents targeting FK506 (e.g., immunophilins, VX-10367, and VX-10428), immunotoxins MAb directed against CD antigen (e.g., XomaZyme-CD5 Plus), leukotriene antagonists (e.g., Sch-40120, Wy-50295, and Wy49232), leukotriene B4 antagonists (e.g., SC-41930, SC-50605, SC-48928, ONO-4057, LB-457, LY-255283, LY-177455, LY-223982, LY-223980, and LY-255253), leukotriene synthesis inhibitors (MK-886, and L-663536), llpase clearing factor inhibitors (e.g., 1-docosanol, and lidakol), lipid encapsulated reducing agent (e.g., Dithranol), liposomal gel (e.g., Dithranol), LO inhibitors (e.g., CD581, CD554, Masoprocol, and Actinex), lithium succinate ointments (e.g., lithium salts, and Efalith), LO/CO inhibitors (e.g., P-8892, P-8977, CHX-108, and FPL-62064), membrane integrity agonists (e.g., lithium salts, and Efalith), microtubule inhibitors (e.g., Posophyliotoxin-containing compound, and Psorex), octapeptide somatostatin analogues (e.g., Lanreotide, angiopeptin, BIM-23014, and Somatuline), oligonucleotides (e.g., ISIS 4730, ISIS 3801, ISIS 1939, and IL-1 inhibitors), peptide agonists (e.g., octapeptide, and peptide T), PKC inhibitors, phospholipase A2 compounds, pospholipase D compounds, photodynamic anticancer agents (e.g., 5-aminolevulinic acid, and 5-ALA), photodynamic therapies (e.g., benzoporphyrin derivative, synthetic chlorins, synthetic porphyrins, and EF-9), photosensitizer (e.g., Porfirmer sodium), PKC inhibitors (e.g., Safingol, and Kynac), platelet activating factor antagonists (e.g., TCV-309), platelet aggregation inhibitors (e.g., CPC-A), prodrug NSAIDs (e.g., G-201), prostaglandin agonist (e.g., elcosapentaenoic acid +gamma-linolenic acid combination, and Efamol Marine), protein inhibitors (e.g., SPC-103600, and SPC-101210), protein kinase C (PKC) inhibitors (e.g., Ro-31-7549, Ro-31-8161, and Ro-31-8220), protein synthesis antagonists (e.g., Calcitriol, Du-026325, LG-1069, LG-1064, AGN-190168, Namirotene, and CBS-211A), purine nucleoside phosphorylase inhibitors (e.g., BCX-34), radical formation agonists (e.g., benzoporphyrin derivative), recombinant antileukoproteinases (e.g., ALP-242), retinoids (e.g., BMY-30123, LG-1069, and LG-1064), retinold derivatives (e.g., AGN-190168), rapamycin binding proteins (FKBP) (e.g., immunophilins, VX-10367, and VX-10428), second generation monoaromatic retinoids (e.g., Acitretin, and Neotigason), soluble IL-1, IL-4 and IL-7 receptors, somatostatin and somatostatin analogues (e.g., Octreotide, and Sandostatin), steroids, (e.g., AGN-191743), streptomyces anulatus isolates (e.g., epocarbazolin-A), superoxide dismutase (e.g., EC-SOD-B), topical formulations (e.g., P-0751, and P-0802), transglutaminase inhibitors, tyrphostin EGF receptor kinase blockers (e.g., AG-18, and AG-555), VCAM-1 inhibitors (e.g., ISIS 3801), vitamin D analogues (e.g., Ro-23-6474, KH-1060, Calcipotriol, BMS-181161, BMY-30434, Dovonex, and Divonex), vitamin D3 analogues (e.g., Tacalcitol, 20 Bonealfa, TV-02 ointment), and vitamin D3 derivatives (e.g., 1,2-diOH-vitamin D3), and the like;

diabetes agents, such as ACE inhibitors (e.g., captopril), amylin, amylin agonists and antagonists (e.g., Normylin™, AC137, GC747, AC253, and AC625), autoimmune compounds (e.g., AI-401), capsaicins (e.g., Zostrix-HP), cell regulators (e.g., protein kinase C inhibitors, and Balanol), domperidones (e.g., Motillum®), fluvastatins (e.g., Lescol), FOX 988, fusion toxins (e.g., DAB389 IL-2, and DAB486 IL-2), gene therapies (e.g., Transkaryotic Therapies), glucagons (e.g., recombinant yeast glucagon), IL-10 compounds, iloprost, immunosuppressives (e.g., tacrolimus, Prograf, and FK-506), proinsulin, insulin and insulin analogs (e.g., AI-401, Nu-Insulin compounds, Humulin, Iletin, Humalog™ LYs-Pro, and Amaryl), insulin-like growth factors (e.g., Chiron/Ciba-Gelgy compounds, Fujisawa compounds, and Genetech compounds), insulinotropins (e.g., Pfizer/Scios Nova compounds), nerve growth factors (e.g., Genentech compounds), oral hypoglycemics (e.g., AS-6, glimepiride, Amaryl, CL 316,243, acarbose, miglitol, recombinant yeast glucagon, GlucaGen™, NovoNorm™ glipizide, insulinotropin, and CI-991/CS-045), platelet-derived growth factors (e.g., Zymo Genetics/Novo Nordisk compounds), sulfonylureas (e.g., tolbutamide, acetohexamide, tolazamide, and chlorpropramide), T cell approaches (e.g., anergize, AnergiX™ Procept compounds, and T cell Sciences compounds), and tolrestats (e.g., Alredase®, and ARI-509), activin, somatostatin, and the like;

stroke agents, such as 5-HT antagonists (e.g., Piperazine derivative), 5-HT reuptake inhibitors (e.g., Milnacipran, and Dalcipran), 5-HT 1A agonists (e.g., SR-57746A, and SR-57746), 5-HT 3 agonists (e.g., SR-57227), 5-HT 4 antagonists, 5-lipoxygenase inhibitors (e.g., low MW dual 5-lipoxygenase and PAF inhibitor CMI-392), ACh agonists (e.g., Pramiracetam, Choline-L-alfoscerate, L-alpha-glycerylphosphoryl-choline, and Delecit), adenosine agonists (e.g., GP-1-4683, ARA-100, and arasine analogs), adenosine AI receptor agonists (e.g., Azaisotere, 2-chloro-N-[4(phenylthio)-1-piperidinyl]adenosine, and 2120136), adenosine reuptake inhibitors (e.g., Diphenyloxazole derivatives), adrenergic transmitter re-uptake inhibitors (e.g., Bifemelane, E-0687, MCI-2016, Alnert, and Celeport), aldose reductase inhibitors (e.g., Spiro-3′ pyrroline derivatives), alpha antagonists (e.g., Drotaverine acephyllinate, and Depogen), alpha 2 agonists (e.g., SNAP-5083, SNAP-5608, and SNAP-5682), AMPA receptor agonists (e.g., heterocyclic compound SYM-1207, and heterocyclic compound SYM-1252), AMPA receptor antagonists (e.g., LY-293558, and LY-215490), Ancrod/Arvin, aspirin, benzothiazoles (e.g., Lubeluzole, and R87926), benzodiazepine receptor antagonists (e.g., 3-oxadiazolyl-1,6-naph-thyridine derivatives, Tetracyclic imidazodiazepineseries imidazenil, FID-02-023, and Ro-23-1412), blood substitutes, bradykinin antagonists (e.g., CP-0127, Bradycor, and Septicor), C5a release inhibitors (e.g., protein derivative CMI-46000), calcium antagonists (e.g., Lemildipine, NB-818, NPK-1886, Trimetazidine derivative, lomerizine KP-2796, Diltiazem analog clentiazem maleate, and TA-3090), calcium channel antagonists (e.g., nitrendipine-like compound diperdipine, YS-201, U-92032, Diltiazem derivative, 1058, SM-6586, KP-840, F-0401, D-31-D, Tetrahydronaphthalene derivatives, fasudil, AT-877, H-7, HA-1044, HA-1077, Eril, darodipine, dazodipine, PY-108-068, Plimo, Dihydropy-ridine, AE 0047, GJ-0956, Lacidipine, GR-43659, GR-43659X, GX-1048, S-312-d, S-312, S-830312, Nilvadipine, and FK-235), calpain inhibitors (e.g., AK-275, and CX-275), carnitine palmitoyl-transferase inhibitors, carvedilol, cerebral calcium antagonist vasodilators (e.g., Nimodipine, and Nimotop), cholinesterase inhibitors (e.g., indole and indazole derivatives, and Tacrine analog), complement factor inhibitors (e.g., TK9C, protein derivative TP16, compinact A, compinact C, Factor D inhibitors, and soluble, recombinant MCP-based complement inhibitors), complement inhibitors (e.g., sCRI/BRL-55730, and YM-203), coronary vasodilators (e.g., Nicorandil, RP-46417, SG-75, and Adancor), CPC-111, cytidyl diphosphochollne/citicholines, cytokines (e.g., NBI-117), Dexanabiol, dopamine agonists, EAA receptors, endothelin antagonists (e.g., SB 209670), endothelin receptor antagonists, excitatory amino acid agonists (e.g., acylated polyamine analogs, and N-(4-hydroxyphenylpropa-nonyl)-spermine analog), excitatory amino acid antagonists (e.g., Tryptophan, 4,6-disubstituted stroke & kynurenine derivatives, NPC-17742, CPC-701, and CPC-702), glutamate antagonists (e.g., Kainate quisqualate NNC-07-9202, NPC-17742, small molecule CNS-1237, NS-257, NS-072, BW-619C, CGS 19755, Riluzole, PK-26124, and RP 54274), glutamate receptor antagonists (e.g., Araxin compounds, Quinoxaline derivative, YM-90K, and YM-900), glycine antagonists, glycine NMDA agonists (e.g., 3-hydroxy-2,5-dioxo-1H-benz[b]azepines), glycine NMDA associated antagonists (e.g., 5,6-dihydro-1H-pyrrolo[1,2,3-de]quinoxaline-2,3-diones, Strychnine-insensitive glycine binding site of NMDA receptor L-687414, Glystasins, ACEA-2011, ACEA-3031, AC-1021, ACPC, and eliprodil), growth factor antagonists (e.g., non-peptide indolocarbazole neutrophic molecules, and CEP-075), GPIIb/IIIa antagonists (e.g., Peptide C68-22), hemorheological agents (e.g., Drotaverine acephyllinate, and Depogen), heparin, hydroxyl radical formation inhibitors (e.g., homopiperazine derivative K-7259), hypocalcemic agents (e.g., calcitonin peptide, related to hCGRP peptide), hypothermic agents/BMY-20862, ICAM-1 compounds (e.g., Enlimomab), immunosuppressants (e.g., small molecule compounds, and NBI-117), integrin general antagonists (e.g., monoclonal antibody AN-100225, and monoclonal antibody AN-100226), interleukin-1 antagonists (e.g., cyclic nitrones), iron-dependent lipid peroxidation inhibitors (e.g., 2-(amino-methyl) chromans), lactic acid accumulation/inhibitors (e.g., small molecule CPC-211), Leukotriene B4 antagonists (e.g., Ebselen, DR-3305, PZ-25, PZ-51, RP 60931, and RP 61605), lipid peroxidase inhibitors (e.g., Idebenone, and Avan), low molecular weight small molecules, methyltransferase stimulants (e.g., 4-methyl benzenesulfonate, ademetionine sulfate tosilate, FO-156, and Ceritan), monoamine oxidase B inhibitors (e.g., MD-280040, MD-200243, MD-280080, Lazabemide, and Ro-19-6327), MS-153, MS-424, /Na+/H+Na+/Li+ exchange inhibitors (e.g., Pyrazine derivatives), nadroparin (e.g., Fraxiparin), nafronyl/naftidrofuryl (e.g., Praxilene), nerve growth factor agonists (e.g., small molecule compounds, CNTF, BDNF, 2.5S NGF, monosialoganglioside GM1, and Sigen/Sygen), neuronal calcium channel blockers (e.g., CPC-304, and CPC-317), neuronal differentiation compounds (e.g., F-spondin), neuropeptide agonists (e.g., Neurotrophic Peptide Trofexin), neutrophil inhibitory factors (e.g., small molecule compounds), nitric oxide agonists (e.g., hydroxy derivative N-3393, hydroxy derivative N-3398, nicorandil, and Therapicon), nitric oxide antagonists, NMDA antagonists (e.g., Spiroisoindoles/dizocilpine derivatives, Oxindole compound, CP-112116, LY-104658, LY-235959, FR-115427, Sialic acid derivative, N-palmitoyl-Betaethylglycoside neuraminic acid, ND-37, Ro-01-6794, 706, Dextrorphan, Ifenprodil analogue eliprodil, SL-82.0715, Lipophilic molecules, HU-211, Remacemide, 934-423, 12495, 12859, 12942AA, Selfotel, CGS-19755, SDZ-EAA-494, CGP-40116, CGP-37849, CGP-39551, and CGP-43487), NMDA antagonist-partial agonists (e.g., Conantokin G peptide SYM-1010), NMDA channel blockers (e.g., Aptiganel, CERESTAT, and CNS 1102), NMDA receptor antagonists, NMDA receptor subtypes (e.g., Kainate quisqua-late NNC-07-9202), non-competitive NMDA antagonists (e.g., FPL-15896), non-ionic copolymer RheothRx, nootropic/acetylcholine agonists (e.g., Oxiracetam, CT-848, and Neuractiv), norepinephrine inhibitors (e.g., Midalci-pran), N-type calcium channel antagonists (e.g., NS-626, and NS-638), opioid antagonists (e.g., Nalmefene, nalmetrene, JF-1, ORF-11676, Cervene, and Incystene), opioid kappa receptor agonists (e.g., acrylacetamide enadoline, and CI-997), organoselenims (e.g., Ebselen, DR-3305, PZ-25, PZ-51, RP 60931, and RP 61605), oxygen scavengers (e.g., Tirilazad mesylate, Lazaroids, and Freedox), PA2 inhibitors (e.g., phospholipase A2 inhibitor), PAF antagonists (e.g., nupafant, and BB-2113), partial glycine NMDA agonists (e.g., ACPC), peptide/GPIIb/IIIa antagonists (e.g., Integrelin), peptidic neuron-specific calcium channel antagonists (e.g., SNX-111), phosphodiesterase inhibitors (e.g., Xanthine derivatives, propentofylline, Hoe-285, and Hextol), phospholipase A2 inhibitors (e.g., small organic molecule CEP-217), plasminogen activators (e.g., r-ProUK (recombinant pro-urokinase), platelet-activating factor antagonists (e.g., UK-74505), platelet adhesion inhibitors (e.g., Peptide), platelet aggregation antagonists (e.g., cilostazol, peptide agents, GPHb-IIIA inhibitor, and TP-9201), platelet aggregation inhibitors (e.g., Diaminoalkanioic acid derivatives), potassium channel agonists (e.g., Nicorandil, RP-46417, SG-75, and Adancor), prolyl endopeptidase (PEP) inhibitors (e.g., JTP-4819), protein kinase C inhibitors (e.g., monosialoganglioside derivative Liga-20), proteolytic enzyme inhibitors (e.g., Protease nexin-1, Incyte, PN-1, PN-2, Nafamostat, FUT-175, Duthan, and Futhan), pyrimidine derivatives, Quinolizine derivatives (e.g., KF-17329, and KF-19863), radical formation antagonists (e.g., EPC-K1), recombinant tissue plasminogen activators (e.g., alteplase, and Activase), Schwann cell derived molecules/promoters, sigma antagonists (e.g., Sigma ligand), sigma receptor antagonists (e.g., tetrahyropyridinyl-isoxazolines and isoxazoles PD-144418), sodium/calcium channel modulators (e.g., Lifarizine, and RS-87476), sodium channel antagonists, streptokinase (e.g., Streptase), substituted guanadine (e.g., small molecule CNS-1237), superoxide dismutase stimulants (e.g., PEG conjugated enzyme superoxide dismutase/Dismutec, and PEG-SOD), thrombin inhibitors, (e.g., non-peptide), thromboxane synthase inhibitors (e.g., Linotroban, and HN-11500), thyrotropin-releasing hormone agonists (e.g., TRH agonists, Protirelin analogthymoliberin, and RX-77368,), ticlopidine (e.g., Ticlid), TJ-8007, TRH agonists (e.g., Thyrotropin releasing hormones, and JTP-2942), trilazard, urokinase (e.g., Abbokinase), w-conopeptide (e.g., SNX-111), and warfarin (e.g., Coumadin), and the like;

agents useful for the treatment of endometriosis (e.g., LHRH analogs),

agents useful for the treatment of uterine contraction (e.g., oxytocin),

agents useful for the treatment of diuresis (e.g., vasopressin),

agents useful for the treatment of cystic fibrosis (e.g., Dnase (i.e., deoxyribonuclease), SLPI, and the like),

agents useful for the treatment of neutropenia (e.g., GCSF),

agents useful for the treatment of lung cancer (e.g., beta 1-interferon),

agents useful for the treatment of respiratory disorders (e.g., superoxide dismutase),

agents useful for the treatment of ischemia/reperfusion injury (e.g., selectin inhibitors, Irf1, and the like);

nitric oxide synthase inhibitors (e.g., N4-methyl-L-arginine, aminoguanidine, N-(iminoethyl)-L-ornithine, thiocitrulline and other citrulline derivatives, N4-nitro-L-arginine, N4-nitro-L-arginine methyl ester, N4-amino-L-arginine, and other arginine derivatives, isothiourea and its derivatives, and the like,

as well as a variety of other agents, such as acyclovir, alendronate sodium, amlodipine, ampicillin, azelaic acid, azithromycin, beclomethasone, betamethasone, bicalutamide, buspirone, carisoprodol, carvedilol, cefaclor, cefadroxil, cefixime, cefprozil, ceftibuten, cefuroxime axetil, cephalexin, cetirizine hydrochloride, cimetidine, ciprofloxacin, cisapride, clarithromycin, clavulanate, clonazepam, clotrimazole, codeine, conjugated estrogens, cyclobenzaprine, desogestrel, dexrazoxane, diazepam, dicyclomine HCl, digoxin, diltiazem, dirithromycin, doxazosin, doxycycline, enalapril, erythromycin, erythromycin base, erythromycin stearate, estradiol, ethinyl estradiol, ethynodiol diacetate, etodolac, famotidine, fluconazole, fluoxetine, fluvastatin, furosemide, gemfibrozil, glipizide, glyburide, guaifenesin, hydrochlorothiazide, hydrocodone, hydrocortisone, ibuprofen, ibutilide fumarate, indapamide, insulin, ipratropium bromide, ketoconazole, ketoprofen, ketorolac tromethamine, lamivudine, lansoprazole, levonorgestrel, levothyroxine, lisinopril, loracarbef, loratidine, lorazepam, losartan potassium, lovastatin, medroxyprogestrone, methylphenidate, methylprednisolone, metoprolol, metoprolol tartrate, moexipril hydrochloride, mometasone furoate, mupirocin, mycophenolate mofetil, nabumetone, nalmefene hydrochloride, naproxen, neomycin, nifedipine, nisoldipine, nitrofurantoin, nizatidine, norethindrone, norgestrel, nortriptyline, ofloxacin, omeprazole, oxaprozin, oxycodone, paroxetine, penicillin, pentoxifylline, phenylpropanolamine, phenytoin, polymyxin, porfimer sodium, potassium chloride, pravastatin, prednisone, promethazine, propoxyphene, pseudoephedrine, quinapril, ramipril, ranitidine, riluzole, salmeterol, saquinavir mesylate, sertraline, sevoflurane, simvastatin, sucralfate, sulfamethoxasole, sumatriptan, temazepam, terazosin, terconazole, terfenadine, tetracycline, theophylline, timolol, tramadol, tramadol hydrochloride, tretinoin, triamcinolone acetonide, triamterene, trimethoprim, valproic acid, venlafaxine, verapamil, wafarin, zolpidem, and the like.

### V—Formulations

mTOR inhibitors and capecitabine may be co-administered according to the present invention in such amounts and for such a time as is necessary or sufficient to achieve at least one desired result. For example, the agents can be administered in such amounts and for such a time that it reduces tumor size, inhibits tumor growth or metastasis, treats various leukemias, delays the progression of disease and/or prolongs the survival time of mammals (including humans) with those diseases or otherwise yields clinical benefit.

In certain cases, an mTOR inhibitor (e.g., AP23573), capecitabine, and physiologically acceptable carrier or excipient are combined in one or more preparations for simultaneous, separate, or sequential administration of the mTOR inhibitor and capecitabine. The agents may be formulated together to provide a composition comprising both agents, or they may be formulated separately, to provide for separate administration, e.g., in the case of staggered administration of the two agents.

Pharmaceutical compositions, according to the present invention, may be administered using any amount and any route of administration effective for achieving the desired therapeutic effect.

The exact amount of pharmaceutical composition to be administered will vary from subject to subject, depending on the species, age, and general condition of the subject, the severity of the condition, and the like (see below).

The optimal pharmaceutical formulation can be varied depending upon the route of administration and desired dosage. Such formulations may influence the physical state, stability, rate of in vivo release, and rate of in vivo clearance of the administered compounds.

The pharmaceutical compositions of the present invention may be formulated in dosage unit form for ease of administration and uniformity of dosage. The expression “unit dosage form”, as used herein, refers to a physically discrete unit of mTOR inhibitor alone, capecitabine alone, or combination of mTOR inhibitor and capecitabine (with or without one or more additional agents) with which a patient may be treated. It will be understood, however, that the total daily usage of the compositions of the present invention will be decided by the attending physician within the scope of sound medical judgment.

After formulation with one or more appropriate physiologically acceptable carrier(s) or excipient(s) in a desired dosage, the pharmaceutical compositions of the present invention can be administered to humans or other mammals by any suitable route. Various delivery systems are known and can be used to administer the inventive compositions, including, tablets, capsules, injectable solutions, encapsulation in liposomes, microparticles, microcapsules, etc. Methods of administration include, but are not limited to, dermal, intradermal, intramuscular, intraperitoneal, intravenous, subcutaneous, intranasal, pulmonary, epidural, ocular, and oral routes. An inventive composition may be administered by any convenient or otherwise appropriate route, for example, by infusion or bolus injection, by absorption through epithelial or mucocutaneous linings (e.g., oral, mucosa, rectal and intestinal mucosa, etc) and may be administered together with other biologically active agents. Administration can be systemic or local. For treatment of nasal, bronchial or pulmonary conditions, preferred routes of administration may be oral, nasal, or via a bronchial aerosol or nebulizer. As will be appreciated by those of ordinary skill in the art, active ingredients of the inventive compositions (e.g., AP23573 and capecitabine) can be administered by the same route (e.g., both intravenously or both orally) or by different routes (e.g., one intravenously, the other orally).

Injectable preparations, for example, sterile injectable aqueous or oleaginous suspensions may be formulated according to the known art using suitable dispersing or wetting agents, and suspending agents. The sterile injectable preparation may also be a sterile injectable solution, suspension or emulsion in a non-toxic parenterally acceptable diluent or solvent, for example, as a solution in 2,3-butanediol. Among the acceptable vehicles and solvents that may be employed are water, Ringer's solution, U.S.P. and isotonic sodium chloride solution. In addition, sterile, fixed oils are conventionally employed as a solution or suspending medium. For this purpose any bland fixed oil can be employed including synthetic mono- or di-glycerides. Fatty acids such as oleic acid may also be used in the preparation of injectable formulations. Sterile liquid carriers are useful in sterile liquid from compositions for parenteral administration.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. Liquid pharmaceutical compositions which are sterile solutions or suspensions can be administered by, for example, intravenous, intramuscular, intraperitoneal or subcutaneous injection. Injection may be via single push or by gradual infusion (e.g., 30 minute intravenous infusion). Where necessary, the composition may include a local anesthetic to ease pain at the site of injection.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming micro-encapsuled matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations can also be prepared by entrapping the drug in liposomes or microemulsions which are compatible with body tissues.

Liquid dosage forms for oral administration include, but are not limited to, pharmaceutically acceptable emulsions, microemulsions, solutions, suspensions, syrups, elixirs, and pressurized compositions. In addition to the active ingredients (e.g., AP23573 and capecitabine), the liquid dosage form may contain inert diluents commonly used in the art such as, for example, water or other solvent, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cotton seed, ground nut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, suspending agents, preservatives, sweetening, flavoring, and perfuming agents, thickening agents, colors, viscosity regulators, stabilizers or osmo-regulators. Suitable examples of liquid carriers for oral administration include water (partially containing additives as above; e.g., cellulose derivatives, such as sodium caboxymethyl cellulose solution), alcohols (including monhydric alcohols and polyhydric alcohols such as glycols) and their derivatives, and oils (e.g., fractionated coconut oil and arachis oil)). For pressurized compositions, the liquid carrier can be halogenated hydrocarbon or other pharmaceutically acceptable propellant.

Solid dosage forms for oral administration include, for example, capsules, tablets, pills, powders, and granules. In such solid dosage forms, the active ingredient(s) is/are mixed with at least one inert, physiologically acceptable excipient or carrier such as sodium citrate or dicalcium phosphate and one or more of: (a) fillers or extenders such as starches, lactose, sucrose, glucose, mannitol, and silicic acid; (b) binders such as, for example, carboxymethylcellulose, alginates, gelatin, polyvinylpyrrolidone, sucrose, and acacia; (c) humectants such as glycerol; (d) disintegrating agents such as agar-agar, calcium carbonate, potato or tapioca starch, alginic acid, certain silicates, and sodium carbonate; (e) solution retarding agents such as paraffin; (f) absorption accelerators such as quaternary ammonium compounds; (g) wetting agents such as, for example, cetyl alcohol and glycerol monostrearate; (h) absorbents such as kaolin and bentonite clay; and (i) lubricants such as talc, calcium stearate, magnesium stearate, solid polyethylene glycols, sodium lauryl sulfate, and mixtures thereof. Other exciplents suitable for solid formulations include surface modifying agents such as non-ionic and anionic surface modifying agents. Representative examples of surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminum silicate, and triethanolamine. In the case of capsules, tablets and pills, the dosage form may also comprise buffering agents. The amount of solid carrier per solid dosage form will vary widely but preferably will be from about 25 mg to about 1 g.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. They may optionally contain opacifying agents and can also be of a composition such that they release the active ingredient(s) only, or preferentially, in a certain part of the intestinal tract, optionally, in a delayed manner. Examples of embedding compositions which can be used include polymeric substances and waxes.

In certain embodiments, it may be desirable to administer an inventive composition locally to an area in need of treatment. This may be achieved, for example, and not by way of limitation, by local infusion during surgery, topically application, by injection, by means of a catheter, by means of suppository, or by means of a skin patch or stent or other implant.

For topical administration, the composition is preferably formulated as a gel, an ointment, a lotion, or a cream which can include carriers such as water, glycerol, alcohol, propylene glycol, fatty alcohols, triglycerides, fatty acid esters, or mineral oil. Other topical carriers include liquid petroleum, isopropyl palmitate, polyethylene glycol, ethanol (95%), polyoxyethylenemonolaurate (5%) in water, or sodium lauryl sulfate (5%) in water. Other materials such as antioxidants, humectants, viscosity stabilizers, and similar agents may be added as necessary. Percutaneous penetration enhancers such as Azone may also be included.

In addition, in certain instances, it is expected that the inventive compositions may be disposed within transdermal devices placed upon, in, or under the skin. Such devices include patches, implants, and injections which release the compound onto the skin, by either passive or active release mechanisms. Transdermal administrations include all administrations across the surface of the body and the inner linings of bodily passage including epithelial and mucosal tissues. Such administrations may be carried out using the present compositions in lotions, creams, foams, patches, suspensions, solutions, and suppositories (rectal and vaginal).

Transdermal administration may be accomplished through the use of a transdermal patch containing the active ingredient(s) and a carrier that is non-toxic to the skin, and allows the delivery of the ingredient(s) for systemic absorption into the bloodstream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient(s) may also be suitable. A variety of occlusive devices may be used to release the active ingredient(s) into the bloodstream such as a semi-permeable membrane covering a reservoir containing the active ingredient(s) with or without a carrier, or a matrix containing the active ingredient.

Suppository formulations may be made from traditional materials, including cocoa butter, with or without the addition of waxes to alter the suppository's melting point, and glycerin. Water soluble suppository bases, such as polyethylene glycols of various molecular weights, may also be used.

Materials and methods for producing various formulations are known in the art and may be adapted for practicing the subject invention. For formulations of rapamycin derivatives or analogs, such as AP23573, see, for example, U.S. Pat. Nos. 5,182,293 and 4,837,311 (tablets, capsules and other oral formulations as well as intravenous formulations) and U.S. Pat. No. 5,516,770 (illustrative formulation for IV administration) and U.S. Pat. Nos. 5,536,729 and 5,559,121 (illustrative formulation for oral administration); U.S. Pat. No. 5,145,684 (nanoparticles) and U.S. Pat. No. 5,989,591 (solid dosage forms) and WO 98/59358.

### VI—Dosage and Administration

A treatment according to the present invention may consist of a single dose or a plurality of doses over a period of time. Capecitabine may be administered concurrently with administration of the mTOR inhibitor. Alternatively or additionally, capecitabine and the mTOR inhibitor may be administered sequentially. For example, capecitabine may be administered prior to or following administration of the mTOR inhibitor (e.g., one or more day(s) before and/or one or more day(s) after.

Administration may be one or multiple times daily, weekly (or at some other multiple day interval) or on an intermittent schedule, all as previously described, with that cycle repeated a given number of times (e.g., 2-10 cycles) or indefinitely.

The administration may be carried out in any convenient manner such as by injection (subcutaneous, intravenous, intramuscular, intraperitoneal, or the like) or oral administration.

Depending on the route of administration, effective doses may be calculated according to the body weight, body surface area, or organ size of the subject to be treated. Optimization of the appropriate dosages can readily be made by one skilled in the art in light of pharmacokinetic data observed in human clinical trials. The final dosage regimen will be determined by the attending physician, considering various factors which modify the action of the drugs, e.g., the drug's specific activity, the severity of the damage and the responsiveness of the patient, the age, condition, body weight, sex and diet of the patient, the severity of any present infection, time of administration, the use (or not) of concomitant therapies, and other clinical factors. As studies are conducted using the inventive combinations, further information will emerge regarding the appropriate dosage levels and duration of treatment.

For additional background information on temsirolimus, see e.g., U.S. Pat. Nos. 2003-0153593 and 2005-0187184 and PCT application No. WO 02/080975. For everolimus, see e.g., WO 03/064383.

IT should also be noted that the combination treatment of this invention may also be used in conjunction with other therapies, including, e.g., surgery, radiotherapy (e.g., gamme-radiation, neuron beam radiotherapy, electron beam radiotherapy, proton therapy, brachytherapy, an systemic radioactive isotopes), endocrine therapy, hyperthermia and cryotherapy.

Alternatively or additionally, methods and compositions of the present invention can be employed together with other agents to attenuate any adverse effects (e.g., antiemetics), and/or with other approved chemotherapeutic drugs, including, but not limited to, alkylating drugs (mechlorethamine, chlorambucil, Cyclophosphamide, Melphalan, Ifosfamide), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), to name a few. For a more comprehensive discussion of updated cancer therapies see, http://www.cancer.gov/, a list of the FDA approved oncology drugs at http://www.fda.gov/cder/cancer/druglistframe.htm, and The Merck Manual, Seventeenth Ed. 1999, the entire contents of which are hereby incorporated by reference.

Methods and compositions of the present invention can also be employed together with one or more further combinations of cytotoxic agents as part of a treatment regimen, wherein the further combination of cytotoxic agents is selected from: CHOPP (cyclophosphamide, doxorubicin, vincristine, prednisone, and procarbazine); CHOP (cyclophosphamide, doxorubicin, vincristine, and prednisone); COP (cyclophosphamide, vincristine, and prednisone); CAP-BOP (cyclophosphamide, doxorubicin, procarbazine, bleomycin, vincristine, and prednisone); m-BACOD (methotrexate, bleomycin, doxorubicin, cyclophosphamide, vincristine, dexamethasone, and leucovorin); ProMACE-MOPP (prednisone, methotrexate, doxorubicin, cyclophosphamide, etoposide, leucovorin, mechloethamine, vincristine, prednisone, and procarbazine); ProMACE-CytaBOM (prednisone, methotrexate, doxorubicin, cyclophosphamide, etoposide, leucovorin, cytarabine, bleomycin, and vincristine); MACOP-B (methotrexate, doxorubicin, cyclophosphamide, vincristine, prednisone, bleomycin, and leucovorin); MOPP (mechloethamine, vincristine, prednisone, and procarbazine); ABVD (adriamycin/doxorubicin, bleomycin, vinblastine, and dacarbazine); MOPP (mechloethamine, vincristine, prednisone and procarbazine) alternating with ABV (adriamycin/doxorubicin, bleomycin, and vinblastine); MOPP (mechloethamine, vincristine, prednisone, and procarbazine) alternating with ABVD (adriamycin/doxorubicin, bleomycin, vinblastine, and dacarbazine); ChIVPP (chlorambucil, vinblastine, procarbazine, and prednisone); IMVP-16 (ifosfamide, methotrexate, and etoposide); MIME (methyl-gag, ifosfamide, methotrexate, and etoposide); DHAP (dexamethasone, high-dose cytaribine, and cisplatin); ESHAP (etoposide, methylpredisolone, high-dose cytarabine, and cisplatin); CEPP(B) (cyclophosphamide, etoposide, procarbazine, prednisone, and bleomycin); CAMP (lomustine, mitoxantrone, cytarabine, and prednisone); CVP-1 (cyclophosphamide, vincristine, and prednisone), ESHOP (etoposide, methylpredisolone, high-dose cytarabine, vincristine and cisplatin); EPOCH (etoposide, vincristine, and doxorubicin for 96 hours with bolus doses of cyclophosphamide and oral prednisone), ICE (ifosfamide, cyclophosphamide, and etoposide), CEPP(B) (cyclophosphamide, etoposide, procarbazine, prednisone, and bleomycin), CHOP-B (cyclophosphamide, doxorubicin, vincristine, prednisone, and bleomycin), CEPP-B (cyclophosphamide, etoposide, procarbazine, and bleomycin), and P/DOCE (epirubicin or doxorubicin, vincristine, cyclophosphamide, and prednisone).

### VII—Indications

Compositions and methods of the present invention can be used to treat primary and/or metastatic cancers, and other cancerous conditions. For example, the inventive compositions and methods should be useful for reducing size of solid tumors, inhibiting tumor growth or metastasis, treating various lymphatic cancers, and/or prolonging the survival time of mammals (including humans) suffering from these diseases.

Examples of cancers and cancer conditions that can be treated according to the present invention include, but are not limited to, tumors of the brain and central nervous system (e.g., tumors of the meninges, brain, spinal cord, cranial nerves and other parts of the CNS, such as glioblastomas or medulla blastomas); head and/or neck cancer, breast tumors, tumors of the circulatory system (e.g., heart, medlastinum and pleura, and other intrathoracic organs, vascular tumors, and tumor-associated vascular tissue); tumors of the blood and lymphatic system (e.g., Hodgkin's disease, Non-Hodgkin's disease lymphoma, Burkitt's lymphoma, AIDS-related lymphomas, malignant immunoproliferative diseases, multiple myeloma, and malignant plasma cell neoplasms, lymphold leukemia, myeloid leukemia, acute or chronic lymphocytic leukemia, monocytic leukemia, other leukemias of specific cell type, leukemia of unspecified cell type, unspecified malignant neoplasms of lymphoid, haematopoietic and related tissues, such as diffuse large cell lymphoma, T-cell lymphoma or cutaneous T-cell lymphoma); tumors of the excretory system (e.g., kidney, renal pelvis, ureter, bladder, and other urinary organs); tumors of the gastronintestinal tract (e.g., oesophagus, stomach, small intestine, colon, colorectal, rectosigmold junction, rectum, anus, and anal canal); tumors involving the liver and intrahepatic bile ducts, gall bladder, and other parts of the biliary tract, pancreas, and other digestive organs; tumors of the oral cavity (e.g., lip, tongue, gum, floor of mouth, palate, parotid gland, salivary glands, tonsil, oropharynx, nasopharynx, puriform sinus, hypopharynx, and other sites of the oral cavity); tumors of the reproductive system (e.g., vulva, vagina, Cervix uteri, uterus, ovary, and other sites associated with female genital organs, placenta, penis, prostate, testis, and other sites associated with male genital organs); tumors of the respiratory tract (e.g., nasal cavity, middle ear, accessory sinuses, larynx, trachea, bronchus and lung, such as small cell lung cancer and non-small cell lung cancer); tumors of the skeletal system (e.g., bone and articular cartilage of limbs, bone articular cartilage and other sites); tumors of the skin (e.g., malignant malonoma of the skin, non-melanoma skin cancer, basal cell carcinoma of skin, squamous cell carcinoma of skin, mesothelioma, Kaposi's sarcoma); and tumors involving other tissues including peripheral nerves and autonomic nervous sytem, connective and soft tissue, retroperitoneoum and peritoneum, eye and adnexa, thyroid, adrenal gland, and other endocrine glands and related structures, secondary and unspefied malignant neoplams of lymph nodes, secondary malignant neoplasm of respiratory and digestive sytems and secondary malignant neosplams of other sites.

More specifically, in certain embodiments of the present invention, inventive compositions and methods are used in the treatment of sarcomas. In some embodiments, the compositions and methods of the present invention are used in the treatment of bladder cancer, breast cancer, chronic lymphoma leukemia, head and neck cancer, endometrial cancer, Non-Hodgkin's lymphoma, non-small cell lung cancer, ovarian cancer, pancreatic cancer, and prostate cancer.

Tumors that can be treated using compositions and methods of the present invention may be refractory to treatment with other chemotherapeuics. The term “refractory”, when used herein in reference to a tumor means that the tumor (and/or metastases thereof), upon treatment with at least one chemotherapeutic other than an inventive composition, shows no or only weak anti-proliferative response (i.e., no or only weak inhibition of tumor growth) after the treatment of such an chemotherapeutic agent—that is, a tumor that cannot be treated at all or only with unsatisfying results with other (preferably standard) chemotherapeutics. The present invention, where treatment of refractory tumors and the like is mentioned, is to be understood to encompass not only (i) tumors where one or more chemotherapeutics have already failed during treatment of a patient, but also (ii) tumors that can be shown to be refractory by other means, e.g., biopsy and culture in the presenceof chemotherapeutics.

Tumors that can be avantageously treated using compositions and methods of the present invention include PTEN-deficient tumors (see, for example, M. S. Neshat et al., PNAS, 2001, 98: 10314-10319; K. Podsypanina et al., PNAS, 2001, 98: 101320-10325; G. B. Mills et al., PNAS, 2001, 98: 10031-10033; M. Hidalgo and E. K. Rowinski, Oncogene, 2000, 19: 6680-6686). As already mentioned above, the FRAP/mTOR kinase is located downstream of the phosphatidyl inositol 3-kinase/Akt-signaling pathway, which is up-regulated in multiple cancers because of loss the the PTEN tumor suppressor gene. PTEN-deficient tumors may be identified, using genotype analysis and/or in vitro culture and study of biopsied tumor samples. Non-limiting examples of cancers involving abnormalities in the phosphatidyl-inositol 3 kinase/Akt-mTOR pathway include, but are not limited to, glioma, lymphoma and tumors of the lung, bladder, ovary, endometrium, prostate or cervix which are associated with abnormal growth factor receptors (e.g., EGFR, PDGFR, IGF-R and IL-2); ovarian tumors which are associated with abnormalities in P13 kinase; melanoma and tumors of the breast, prostate or endometrium which are associated with abnormalities in PTEN; breast, gastric, ovarian, pancreatic, and prostate cancers associated with abnormalities with Akt; lymphoma, cancers of the breast or bladder and head and neck carcinoma associated with abnormalities in eIF-4E; mantle cell lymphoma, breast cancer and head and neck carcinomas associated with abnormalities in Cyclin D; and familial melanoma and pancreas carcinomas associated with abnormalities in P16.

### VIII—Pharmaceutical Packages

In another aspect, the present invention provides a pharmaceutical kit comprising one or more containers (e.g., vials, ampoules, test tubes, flasks or bottles) containing one or more of the ingredients of an inventive pharmaceutical composition, allowing the simultaneous or sequential administration of the mTOR inhibitor and capecitabine.

The different ingredients of a pharmaceutical package may be supplied in a solid (e.g., lyophilized) or liquid form. Each ingredient will generally be suitable as aliquoted in its respective container or provided in a concentrated form. Pharmaceutical packs or kits may include media for the reconstitution of lyophilized ingredients. The individual containers of the kit will preferably be maintained in close confinement for commercial sale.

In certain embodiments, the pharmaceutical package includes one or more additional approved therapeutic agent(s) (e.g., one or more other anti-cancer agents, as described above). Optionally associated with such container(s) can be a notice or package insert in the form prescribed by a governmental agency regulating the manufacture, use or sale of pharmaceutical or biological products, which notice reflects approval by the agency of manufacture, use or sale for human administration. The notice or package insert may contain instructions for use of the pharmaceutical composition according to methods disclosed herein.

### Examples

The following examples describe approaches for practicing the invention. However, it should be understood that these examples are for illustrative purposes only and are not meant to limit the scope of the invention. Furthermore, unless the description in an Example is presented in the past tense, the text, like the rest of the specification, is not intended to suggest that experiments were actually performed or data were actually obtained.

The results with AP23573 reported in Examples 2 and 3 below are presented, as a poster, at the Annual Meeting of the American Society of Clinical Oncology, ASCO 2006, Jun. 2-6, 2006 (A. Perotti, M. Maur, L. Viganò, E. Gallerani, R. Angst, J. Albanell, C. Sessa, R. Laliberte, S. Marsoni, and L. Glanni, “Phase Ib pharmacokinetic (PK) and pharmacodynamic (PD) study to define the optimal dose for combining the mTOR inhibitor AP23573 with Capecitabine (CAPE)”, Abstract 3065). This poster is incorporated herein by reference in its entirety.

### Example 1A

### Oral Formulation of the Rapamycin Analog, AP23573

The following procedure was used to prepare a tablet containing 10 mg of AP23573 and containing the listed components. The tablets are coated with two different coatings—a film-coated tablet for immediate release and an enteric-coated tablet for delayed release. The composition of the core tablet is shown in the following table. Core tablets are film-coated and may be used as such, or may be enteric-coated.

| Component | Weight Percent |
|---|---|
| AP23573 | 8.00% |
| Butylated Hydroxytoluene | 0.08% |
| Hydroxy Propyl Cellulose | 8% |
| Lactose Monohydrate | 50.57% |
| Microcrystalline Cellulose | 30.85% |
| Croscarmellose Sodium | 2.00% |
| Magnesium Stereate | 0.50% |
| Dehydrated Alcohol (Ethanol)* | — |

*Use in processing but does not necessarily appear in final product

Hydroxypropyl Cellulose, Lactose Monohydrate, Microcrystalline Cellulose, and half of the Croscarmellose Sodium, were mixed in a high shear granulator. The AP23573 and Butylated Hydroxytoluene (BHT) were dissolved in Dehydrated Alcohol, USP, mixing not less than 45 minutes. The solution of AP23573 and BHT was added to the granulator and mixed to a wet mass for approximately 3 minutes.

The granulation was dried in a fluid bed dryer at 45-55° C. for 60-90 minutes, after which the dried granulation was passed through a mill fitted with a 0.045-inch screen opening to remove oversized granulation. The milled granulation was then blended with Magnesium Stearate, NF and the remaining half of the Croscarmellose Sodium, NF.

The granulation was pressed into tablets using a tablet press set up with 6 mm round concave tooling. The press was adjusted as required for a target tablet weight of 125.0 mg, hardness of 5.5 kp, friability no more than 1%, and disintegration time less than 10 minutes.

### Film Coating

A film coating may be prepared according to following procedure using the following components. The tablets are added to a coating pan and are coated with a solution of Copovidone in Dehydrated Alcohol, USP (20:80 w/w), maintaining a product temperature of 20-35° C., until a weight gain of 5% is achieved. The pan is then cooled and the film-coated tablets allowed to dry. Film-coated tablets may be packaged as such, or may be enteric coated.

### Enteric Coating

An enteric coating may be prepared according to following procedure using the following components.

| Film Coating | Percent of Suspension |
|---|---|
| Methacrylic Acid Copolymer | 11.03% |
| Triethyl Citrate | 2.16% |
| Talc | 2.81% |
| Dehydrated Alcohol (Ethanol)* | 84.00% |

*Use in processing but not for retention in final product

For enteric coating, the tablets are placed in a coating pan and coated with a suspension of Methacrylic Acid Copolymer, NF, Triethyl Citrate, NF, and Talc in Dehydrated Alcohol, USP, maintaining a product temperature of 20-35° C., until a weight gain of 8% is achieved. The pan is then cooled, and the enteric-coated tablets allowed to dry.

### Example 1B

### IV Formulation of AP23573

62.5 mg/mL of AP23573 in ethanol is diluted with a diluent comprising 5.2% propylene glycol and 5.2% polysorbate 80 in Water for Injection (WFI). The diluted drug product is further diluted in 0.9% normal saline prior to administration to patients. Prior to dilution it may be kept in storage at −20° C. for at least 6 months. The diluent is recommended for storage at 2-8° C. for at least 6 months.

The diluted AP23573 is prepared for administration to patients in 250 mL of 0.9% normal saline and may be administered to patients by intravenous infusion over a 30-minute period.

### Example 2

### Phase Ib Pharmacokinetic (PK) and Pharmacodynamic (PD) Study to Define the Optimal Dose for Combining the mTOR Inhibitor AP23573 with Capecitabine (CAPE)

### I. Introduction

AP23573 is a novel mTOR inhibitor that has demonstrated single-agent, anti-cancer activity in phase I and phase 2 trials, in a range of solid tumors; dose limiting toxicity was oral mucositis with other dosing schedules. In vitro experiments show that AP23573 is at least additive with several cytotoxics including 5-fluorouracil (5FU). Capecitabine (CAPE) is activated to 5FU by thymidine phosphorylate which may be highly expressed in tumors and correlates with progression through angiogenic mechanisms controlled by mTOR.

Vascular endothelial growth factor (VEGF) is a major angiogenesis growth factor that induces angiogenesis and vasculogenesis in vivo through interaction with the tyrosine kinase receptors VEGFR-1 (flit) and VEGFR-2 (flk-1/KDR) on endothelial cells. mTOR inhibition is associated with decreased VEGF secretion (M. Guba et al., Nature Med., 2002, 8: 128-35). In vitro, AP23573 inhibits VEGF production in tumor cells and growth factor driven proliferation (R. Pollack et al., “Cell shrinkage, cell arrest, and anti-angiogenesis underlie the anti-tumor activity of the mTOR inhibitor AP23573”, Proceedings of the 2003 AACR-NCI-EORTC, #B-160, 2005). Further, reduced VEGF levels have been observed in patients treated with AP23573 (V. M. Rivera et al., “Analysis of potential blomarkers of AP23573 activity in phase II trial in patients with sarcoma, Proceedings of the 2005 AACR-NCI-EORTC, #B-181, 2005). Because of the anti-angiogenic activities of both 5FU and AP23573, it is expected that this combination will be synergistic.

To date, combinations of other mTOR inhibitors with anti-metabolites have not been well tolerated, and excessive toxicity has been reported with these combinations (Punt et al., Ann. Oncol., 2003, 14: 931; Pacey et al., Journal of Clinical Oncology, 2004 ASCO Annual Meeting Proceedings (Post-Meeting Edition). Vol 22, No 14S (July 15 Supplement), 2004: 3120). The present study combining administration of AP23573 with capecitabine in patients with advanced disease was conducted to: (1) define the MTD of AP23573 in combination with CAPE; (2) characterize the safety profile of AP23573 in combination with CAPE; and (3) examine pharmacokinetic and pharmacodynamic characteristics.

### II. Methods

Eligibility Criteria included histo/cyto diagnosis of solid tumors; no documented resistance to fluoropyrimidines (Progresive Disease during or within 6 months after fluoropyrimidine); preferentially≦two prior chemotherapies for advanced disease; ECOG≦1; adequate renal (creatinine 1.5×) hematology and liver function (≦2.5×ULN for AST/ALT; ULN bilirubin); and serum choloesterol<350 mg/dL and triglycerides<400 mg/dL).

Definitions. Dose limiting Toxicity (DLT) is defined by: febrile neutropenia, neutrophils<500×10⁶/L for ≧5 days); ≧Grade 3 (CTC) thrombocytopenia/mucositis; non-haematological toxicities≧Grade 2 (diarrhea, cardiac, skin or renal); missing 2 consecutive weekly doses due to any drug related toxicity. Maximum tolerated dose (MTD) is defined as the dose at which 2 of 3 or 2 of 6 patients experience DLT. Recommended dose (RD) is defined as 1 dose level below MTD. CR stands for Complete Response, and PR for Partial Response.

Clinical Trial. The study reported herein was a multi-center, open-label, uncontrolled Phase 1b trial. At each dose level, 3-6 patients were treated depending on toxicities. The first 3 patients entered the trial simultaneously; subsequent patients entered the trial after having been observed for at least 1 cycle.

Starting doses were AP23573: 25 mg iv on Days 1, 8 and 15 and CAPE: 1650 mg/m2 po daily on Days 1-14, repeating every 28 days. CAPE starting dose of 1650 mg/m2 was increased to 1800 mg/m2 at dose level V.

Planned pharmacokinetic (PK) and pharmacodynamic (PD) studies included: analysis of plasma, peripheral blood mononuclear cells (PBMC), skin, and tumor samples for effects on mTOR associated pathways*, VEGF* levels and the metabolism of CAPE and fluoropyrimidines (results of studies marked with a “*” are not presented herein).

### III. Results

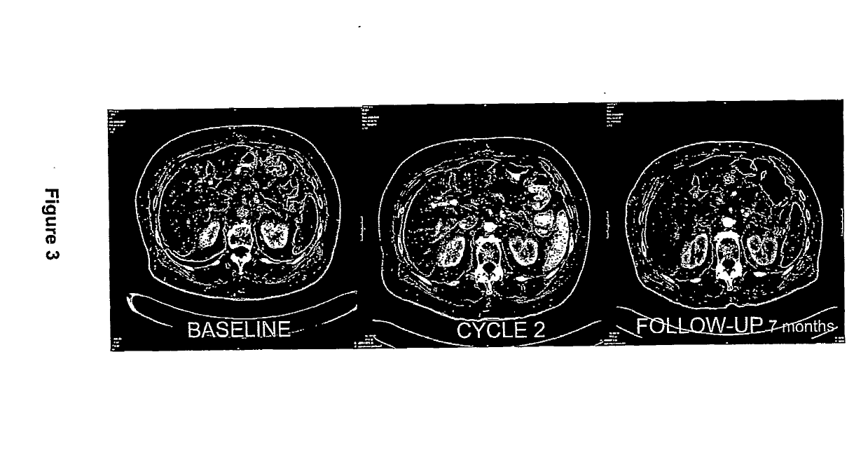

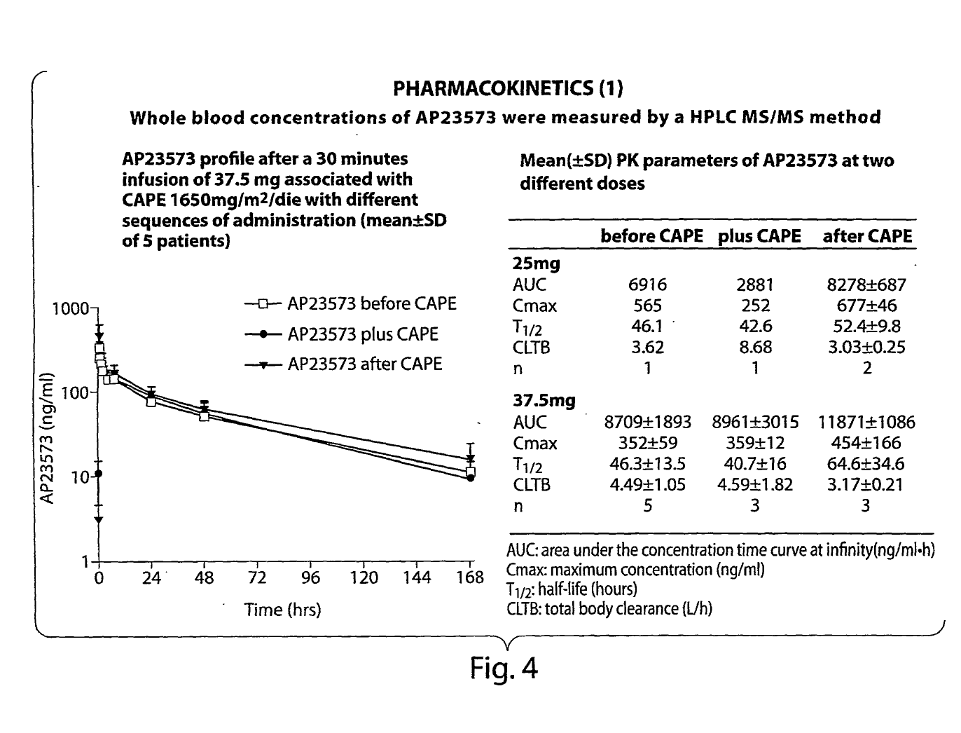

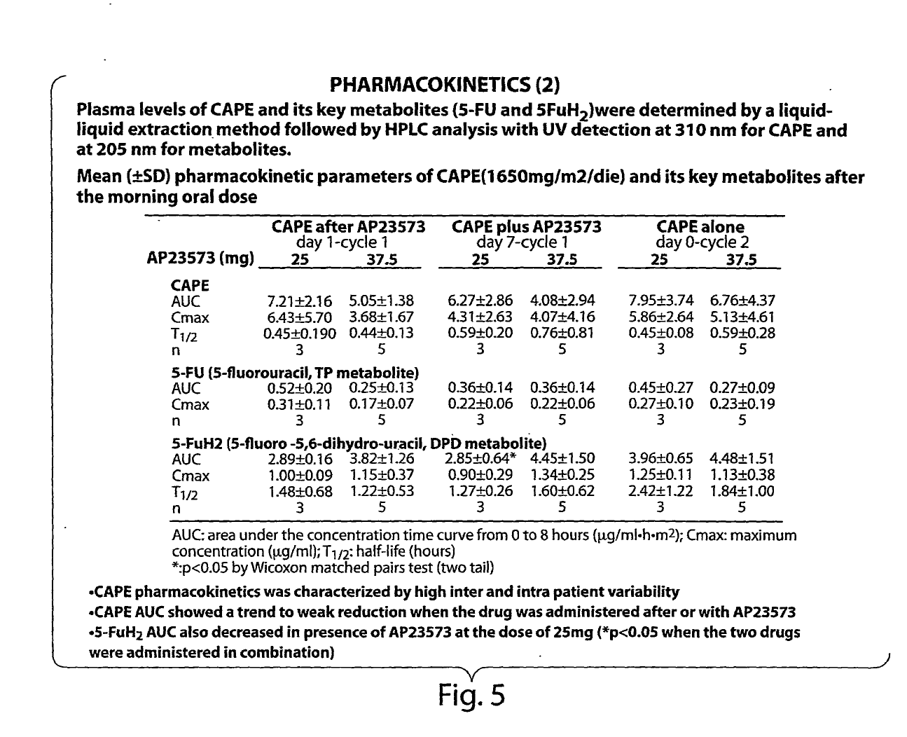

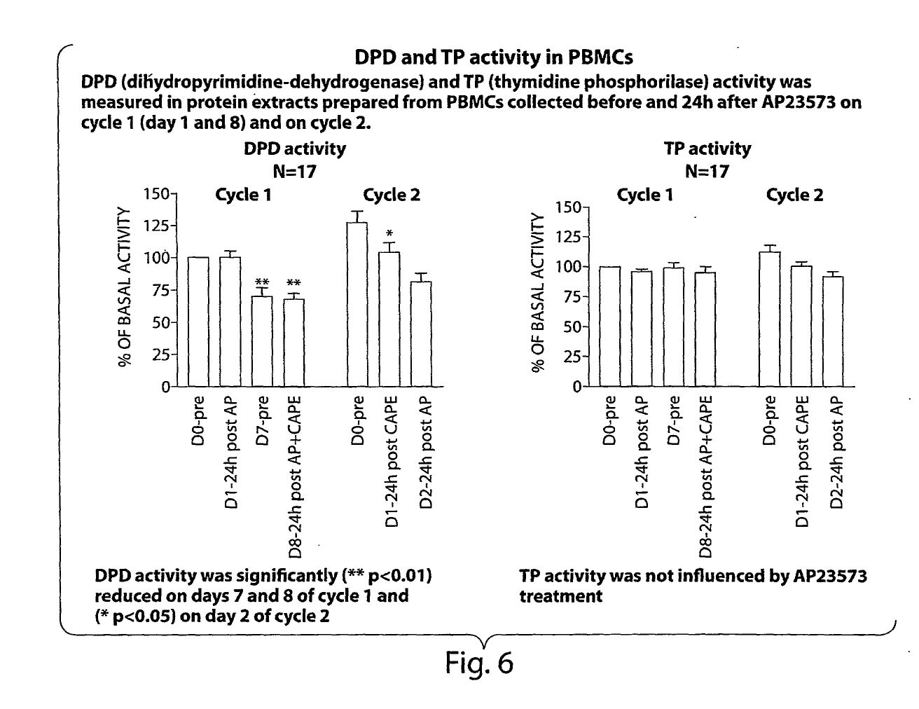

Results obtained are shown on FIGS. 3-6 and Tables 2-4. They show that the AP23573/CAPE combination is generally well tolerated with reversible and manageable side-effects. This result is surprising in light of results obtained in Phase I clinical trials performed by others (Punt et al., Ann. Oncol., 2003, 14: 931; Pacey et al., Journal of Clinical Oncology, 2004 ASCO Annual Meeting Proceedings (Post-Meeting Edition). Vol 22, No 14S (July 15 Supplement), 2004: 3120).

In the present study, the pharmacokinetics of 5FU was found not be impacted by CAPE. Furthermore, the combination elicited a noteworthy antitumor response. This study is the first report of successful combination of a mTOR inhibitor and an anti-metabolite.

### Example 3

### Peripheral Blood Mononuclear Cells Pharmacodynamic Analysis

### I. Methods

Relative phospho-4E-BP1 levels were determined to assess the inhibition of mTOR signaling in PBMCs.

Protein extracts were prepared from PBMCs collected at various times and after dosing with AP23573 and CAPE. Protein extracts were analyzed by Western blotting, in duplicate, using antibodies specific for total-4E-BP1 (Cell Signaling Technology) or phospho-4E-BP1 (Ser65/Thr70: Santa Cruz). Phospho-4E-BP1 levels were normalized to total in each sample and expressed relative to the Day 0 Pre-dose sample.

### II. Results

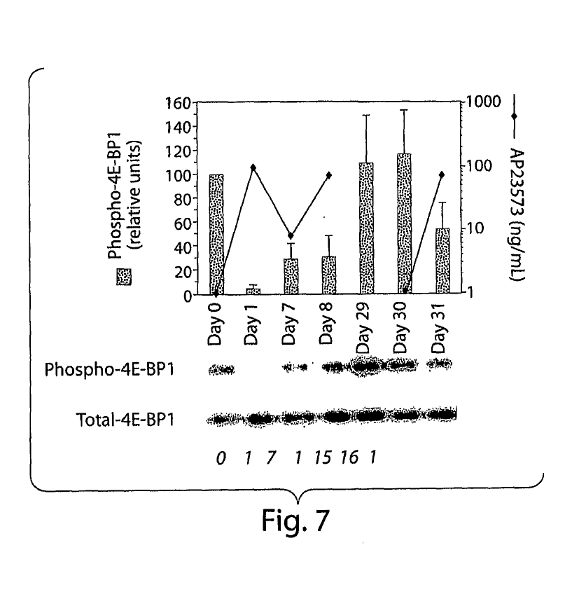

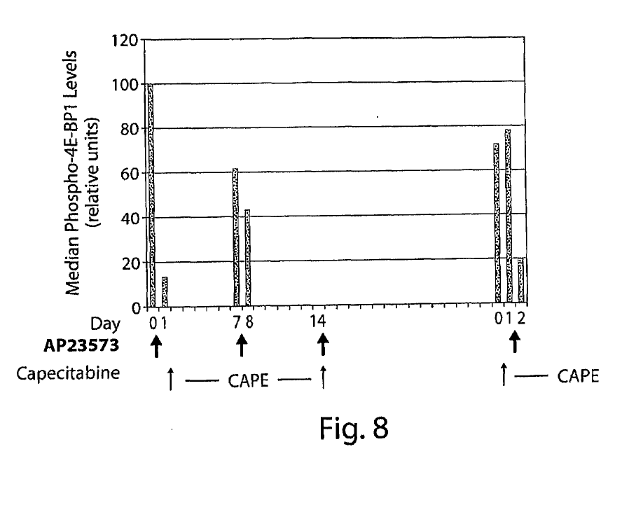

Representative mTOR inhibition in PBMCs following dosing with AP23573 and CAPE is presented on FIG. 7. Preliminary results obtained in this study are summarized on FIG. 8. In FIG. 8, median phospho-4E-BP1 levels from 14 patients analyzed to date are plotted. 24 hours after initial dosing with AP23573, phospho-4E-BP1 levels were found to be reduced to 14% of baseline levels (86% reduction). By Day 7, phospho-4E-BP1 levels were 61% of baseline. 24 hours after the Day 7 dose with AP23573, phospho-4E-BP1 levels were at 43% of baseline (only a 30% reduction from Day 7). At the beginning of cycle 2, 15 days removed from the last AP23573 or capecitabine dose, phospho-4E-BP1 levels were about 75% of baseline levels. 24 hours after dosing with AP23573 phospho-4E-BP1 levels were 19% of baseline (or reduced by 75% relative to C2D1).

### Example 4

### Combination Therapy Using AP23573 (po) and Capecitabine (CAPE) (po) in Treatment of Neoplasms

Dosing begins at month 1, day 1 with AP23573 (40 mg/day po for 5 days each week) and daily capecitabine (po, 1000 or 1250 mg, twice a day).

The CAPE is administered daily typically with food or within 30 minutes after food. CAPE is given for 14 days, followed by 7 or 14 days without CAPE.

Dose adjustments and/or delays for either agent are permitted. If toxicity issues arise at this treatment level in a given patient, dosing with the AP23573 may be delayed, reduced (e.g., to 30 mg/day), dropped from QDx5 to QDx4 for example, or discontinued for a brief period (e.g., 1, 2 or three weeks) during the regimen.

### Example 5

### Combination Therapy Using AP23573, Capecitabine (CAPE) and Tykerb in Treatment of Neoplasms

Dosing begins at month 1, day 1 with AP23573 (40 mg/day po for 5 days each week(QDx5), daily capecitabine (po, 1250 mg, twice a day, QDx7 for 14 days every 21 days, and with 1250 mg Tykerb given daily po (QDx7).

The CAPE is administered daily typically with food or within 30 minutes after food, while the Tykerb is administered at least one hour before or one hour after a meal.

Dose adjustments and/or delays for any of the three agents are permitted. If toxicity issues arise for a patient at a given treatment level, dosing with the AP23573 may be reduced (e.g., from 40 to 30 mg/day), dropped from QDx5 to QDx4 for example, or discontinued for a brief period (e.g., 1, 2 or three weeks) during the regimen. Likewise the dose of CAPE may be reduced from 1250 mg to 1000 mg for example and the holiday extended from one week to two weeks in between courses of CAPE administration (i.e., switching to CAPE administration for 14 days every 28 days). Tykerb dose may also be reduced or discontinued for a brief period.

### Example 6

### Combination Therapy Using Temsirolimus and Capecitabine (CAPE) in Treatment of Neoplasms

Dosing begins at month 1, day 1 with weekly intravenous (iv) temsirolimus (15, 25 or 50 mg/weekly dose) and daily capecitabine (po, 1000 or 1250 mg, bid).

Temsirolimus is administered iv once per week over a 30-minute period using an in-line filter and an automatic dispensing pump. Optionally, antihistamine (diphenhydramine, 25 to 50 mg iv or the equivalent) is administered about 30 minutes prior to temsirolimus infusion.

The CAPE is administered daily typically with food or within 30 minutes after food. CAPE is given for 14 days, followed by 7 or 14 days without CAPE.

Dose adjustments and/or delays for either agent are permitted. For example, dosing with the temsirolimus may be delayed or discontinued for a brief period (e.g., 1, 2 or three weeks) during the regimen. CAPE may be adjusted as in other examples.

### Example 7

### Combination Therapy Using Everolimus (po) and Capecitabine (CAPE) (po) in Treatment of Neoplasms

Dosing begins at month 1, day 1 with everolimus (10 mg/day po QDx7) and daily capecitabine (po, 1000 or 1250 mg, twice a day).

The CAPE is administered daily typically with food or within 30 minutes after food. CAPE is given for 14 days, followed by 7 or 14 days without CAPE.

Dose adjustments and/or delays for either agent are permitted. If toxicity issues arise at this treatment level in a given patient, dosing with everolimus may be delayed, reduced (e.g., to 5 mg/day), dropped from QDx7 to QDx6, or discontinued for a brief period (e.g., 1, 2 or three weeks) during the regimen. CAPE dosing may be adjusted as in other examples.

### Other Embodiments

Other embodiments of the invention will be apparent to those skilled in the art from a consideration of the disclosure of this document. Note however that the specification and examples are intended as exemplary only, with the true scope of the invention being indicated by the following claims.

TABLE 1
PATIENT CHARACTERISTICS

| | |
|---|---|
| Evaluable for safety | 22 |
| Evaluable for first cycle DLTs | 18 |
| Age, median (range) | 61 (28-74) |
| Male/Female | 8/14 |
| ECOG PS 0/1 | 14/8 |
| >2 Prior regimens of chemotherapy | 7 |
| for metastatic disease | |
| Tumor types | |
| Renal | 7 |
| Other Genitourinary | 2 |
| Gyneacological | 7 |
| Colorectal | 1 |
| Advanced Breast Ca. | 1 |
| Head & Neck | 1 |
| Soft Tissue Sarcoma | 3 |

TABLE 2
Dose escalation summary/FIRST CYCLE DLTS

| Dose level | #pts evaluable for DLT | # DLTs |
|---|---|---|
| I | 3 | 0 |
| II | 6 | 1 (G2 skin toxicity) |
| III | 3 | 0 |
| IV | 3 | 0 |
| V | 3 | 2 (1 pt G2 skin toxicity |
| | | the other G3 (mucositis) |

TABLE 3
ALL CYCLE TOXICITY (% pt with drug related toxity)

| | DL I | | | DL II | | DL III | | DL IV | | DL V | |
|---|---|---|---|---|---|---|---|---|---|---|---|
| AE | G1 | G2 | G3 | G1 | G2 | G1 | G2 | G1 | G2 | G1 | G2 |
| ANC | | 33 | 33 | 50 | | 25 | | | | | |
| Platelets | | 66 | | 50 | | 33 | 33 | | | | |
| Skin | 33 | | | 25 | | 33 | 33 | 67 | | 50 | |
| Asthenia | 33 | 33 | | 38 | | 50 | | 33 | 33 | | 25 |
| Mucositis | 33 | | | 38 | 25 | | 75 | | | 25 | 25 |

NO GRADE 4 TOXICITIES HAVE BEEN NOTED

TABLE 4
TUMOR RESPONSE

| | | | | | Duration of |
|---|---|---|---|---|---|
| | | | | | response |
| | | | | | (DR)/Time |
| | | | | | to tumor |
| | DOSE | | CR/PR to prior | BEST RESPONSE | progression |
| Pt# | level | TUMOR | therapy | AP23573/CAPE | (TTP) Mos |
| 005 | II | UTERUS | Yes | PR | 1.2/2.9 |
| 003 | I | RENAL | No | SD | 5.7/7.5 |
| 001 | I | RHINOPHARYNX | Yes | SD | 4.6/6.4 |
| 011 | II | BLADDER | No | SD | 4.2/5.9 |
| 009 | II | RENAL | Yes | SD | 3.1/4.7 |

What is claimed is:

1. A method of treating a cancer in a patient in need thereof, the method comprising administering to the patient an effective amount of an mTOR inhibitor in combination with the administration of capecitabine, in a dose of 1000 to 2500 mg of capecitabine/m²/day.

2. The method of claim 1, in which the capecitabine is administered orally, on a daily schedule for 7-14 days every 21-28 days.

3. The method of claim 1 or 2, in which the daily dose of capecitabine is administered in two separate portions at different times of day.

4. The method of any of claims 1-3, in which Herceptin is also administered to the patient.

5. The method of any of claims 1-3 in which Tykerb or Taxotere is also administered to the patient.

6. The method of any of claims 1-5, in which a the mTOR inhibitor is AP23573, Sirolimus, Everolimus or Temsirolimus.

7. The method of any of claims 1-6, wherein the cancer is a cancer of the prostate, endometrium, breast, ovary, cervix, uterus, head and neck, lung (small cell and non-small cell), pancreas, kidney, brain, colorectum, bladder, mouth, larynx, esophagus or stomach; a sarcoma, melanoma, multiple myeloma, B-cell lymphoma, mantle cell lymphoma, Non-Hodgkin's Lymphoma, or leukemia.

8. The method of any of claims 1-7, wherein the mTOR inhibitor is administered orally.

9. The method of claim 8, wherein the mTOR inhibitor is administered orally in a dose of 2-160 mg/day on one or more days per week.

10. The method of claim 9 wherein the the mTOR inhibitor is AP23573, sirolimus or everolimus.

11. The method of any of claims 1-7, wherein the mTOR inhibitor is administered parenterally.

12. The method of claim 11 wherein the the mTOR inhibitor is AP23573, sirolimus or temesirolimus.

13. A composition comprising 2 to 50 mg of an mTOR inhibitor and 500-5000 mg of capecitabine and at least one physiologically acceptable carrier or excipient.

14. A pharmaceutical kit comprising an mTOR inhibitor and capecitabine in one or more unit dosage forms for simultaneous, separate or sequential use in the treatment of a cancer in a subject.

15. The pharmaceutical kit of claim 14, in which the mTOR inhibitor and the capecitabine are formulated for oral administration.

16. The pharmaceutical kit of claim 14, in which the the capecitabine is formulated for oral administration and the mTOR inhibitor is formulated for parenteral administration and is optionally accompanied by a container of diluent.

17. The use of an mTOR inhibitor in the preparation of a medicament which may be administered, for the treatment of a cancer, in a combination therapy with capecitabine, in a dose of 1000 to 2500 mg of capecitabine/m²/day.

18. The use of capecitabine in the preparation of a medicament which may be administered in a dose of 1000 to 2500 mg of capecitabine/m²/day for the treatment of a cancer, in a combination therapy with an mTOR inhibitor.

19. The use of claim 17 or 18 in which the mTOR inhibitor is AP23573, sirolimus, everolimus or temsirolimus.

20. The use of claim 17, 18 or 19 in which the combination therapy comprises a method of any of claims 1-5 or 7-12.

21. The method of claim 12 wherein the AP23573 is administered as a 15-60 minute intravenous infusion.
